(19) United States
(12) Patent Application Publication
(10) Pub. No.: US 2012/0255355 A1
(43) Pub. Date: Oct. 11, 2012

(54) HAND MUSCLE MEASUREMENT DEVICE

(76) Inventors: Shuai Xu, Boston, MA (US); Gloria R. Gogola, Houston, TX (US); Graham Sattler, Houston, TX (US); Sridhar Madala, Houston, TX (US); Robert LiKamWa, Houston, TX (US)

(21) Appl. No.: 13/500,607

(22) PCT Filed: Oct. 8, 2010
(86) PCT No.: PCT/US10/52085

§ 371 Date: Jun. 25, 2012

Related U.S. Application Data
(60) Provisional application No. 61/249,925, filed on Oct. 8, 2009.

(51) Int. Cl. A61B 5/22

(52) U.S. Cl. 73/379.02

(57) ABSTRACT

The present disclosure relates to an integrated system for measuring hand strength and dexterity. Specifically, the integrated system allows for measuring of hand muscle strength through the pinch-grip test, of intrinsic hand muscle strength, and testing of performance in various dexterity tests.

### CROSS-REFERENCE TO RELATED APPLICATIONS

This application claims priority to U.S. Provisional Application No. 61/249,925, filed on Oct. 8, 2009 and entitled TESTING MUSCULAR STRENGTH AND DEXTERITY, the disclosure of which is incorporated herein by reference.

### FIELD OF INVENTION

The present invention generally relates to measuring muscle strength, and more particularly to devices and methods for administering the pinch-grip, intrinsic muscle strength and/or dexterity tests.

### BACKGROUND

There are different tests that can be performed on a person's hand to obtain measurements of the performance of various hand muscles. The results can be used for at least diagnostic purposes. For example, medical professionals have used the pinch-grip test for the past four decades to evaluate upper-extremity function. One application of the pinch-grip test is to allow doctors to objectively monitor the progress of patients who have suffered hand injury, either through accidents or debilitating diseases such as arthritis, or who have undergone hand surgery.

Another conventional hand muscle test is the dexterity test, which has been used to assess manipulative abilities of the hands and an overall cognitive function. The dexterity test may aid in monitoring the progress of patients who have suffered loss of grasping strength and dexterity that may result from old age and various other health related causes such as injuries or diseases.

More recently, there have been great interests in obtaining reliable measurements of intrinsic hand muscle strength (IHMS), which may not be properly measured by the pinch-grip and dexterity tests. Intrinsic hand muscles are important for dexterity and precision movements. Intrinsic hand muscles are located within the hand itself and account for planar lateral movement of the fingers and abduction, adduction, and flexion of the thumb. As previously mentioned, the intrinsic hand muscles differ from the extrinsic muscles, which are measured through pinch-grip motion. Hand trauma, rheumatoid arthritis, congenital hand defects and a host of serious pathologies, such as neurological diseases, correlate to a decrease in intrinsic hand muscle strength. Beyond blunt trauma, the assessment of IHMS is necessary for diseases such as carpal tunnel syndrome. Examples of a device for measuring IHMS are disclosed in PCT Application No. PCT/US10/28837, which claims priority to U.S. provisional Application No. 61/164,271, the disclosures of which are incorporated herein by reference.

Accordingly, the pinch-grip, intrinsic muscle strength, and dexterity tests are useful tools for medical professionals. However, devices currently available to medical professionals are only capable of and designed for administering one type of test. That is, if a medical professional wants to administer the pinch-grip test and measure IHMS of a patient, she would need at least two different devices, one for the pinch grip test and the other to measure IHMS. In view of the above, there is a need for an integrated device that has the ability to provide pinch/grip/intrinsic muscle strength and dexterity measurements in one system.

### SUMMARY OF THE INVENTION

One objective of the present disclosure is to provide an integrated device that allows a user to perform a combination of strength and dexterity measurements of a patient's hand.

Another objective of the present disclosure is to provide a device that allows a user to reliably measure intrinsic muscle strength.

Yet another objective of the present disclosure is to provide an integrated device that allows for measurement of intrinsic muscle strength and other hand muscle strength via the pinch and grip test and measure.

Still another objective of the present disclosure is to provide a device that allows for measurement of other muscles in the body, such as muscles in the arm of a patient.

Another objective of the present disclosure is to provide a device to assist in the rehabilitation of a patient's hand or arm injury.

Yet another objective of the present disclosure is to provide a force sensor system that can determine the type of force being applied from the force itself instead of manual selection of the type of force by a user.

Still another objective of the present disclosure is to provide a dexterity testing device that can provide sensing of whether an object is in a peg hole and automatic timing of a dexterity test.

To meet the above objectives, there is provided, in accordance with one aspect of the present disclosure, a device comprising a strength testing unit. The strength testing unit comprising a force sensor, a front handle configured to receive the force sensor, a first fastener, a second fastener, where the first and second fasteners are coupled to the force sensor, a back handle coupled to the first and second fasteners and positioned at a distance away from the front handle; a force transfer member coupled to the back handle, where the force sensor is configured to generate one or more electric signals corresponding to the force applied to at least one of the front and back handles; and a processing unit coupled to the force sensor, the processing unit configured to determine a measurement of the applied force and to display the measurement on a first display coupled to the processing unit.

In some embodiments, the device further includes a board comprising one or more holes, one or more sensors coupled to the processing unit, the one or more sensors configured to detect whether one or more objects are inside the one or more holes and to generate one or more electric signals in response to said detection; and one or more openings configured to receive the strength testing unit. In some embodiments, the one or more sensors are configured to detect photons.

In some other embodiments, the device further includes a second display configured to communicate with the processing unit to at least receive and display information transmitted from the processing unit; and a data storage unit configured to communicate with the processing unit to at least receive and store information transmitted from the processing unit.

In yet some other embodiments, the device further includes a timing unit coupled to the processing unit, the timing unit configured to measure the time elapsed between at least two electric signals generated by the one or more sensors.

In some embodiments, the processing unit determines a type of test being administered based, at least in part, on the type of force being applied to the force sensor. And in other embodiments, the processing unit provides to a user information associated with a force being applied to the device by the user, said information comprising an effective range of force to allow the user to maximize the force being applied to the device within said range.

In accordance with another aspect of the present disclosure, there is provided a device comprising a strength testing unit. The testing unit comprises a pinch tester comprising a first component coupled to a first force sensor, a second component, where the second component is immobile, an intrinsic muscle tester comprising a force transfer member configured to fit on or around one or more digits of a user's hand, wherein the force transfer member is coupled to the first force sensor, where the first force sensor is configured to generate one or more electric signals corresponding to the force applied to at least the first component of the pinch tester or the intrinsic muscle tester; and a processing unit coupled to the first force sensor, the processing unit configured to determine a measurement of the applied force and to display the measurement on a display coupled to the processing unit.

In some embodiments, the device further includes a grip tester comprising a front component coupled to a second force sensor, where the second force sensor is configured to generate one or more electric signals corresponding to the force applied to at least the front component; and a back component located a distance away from the front component, said distance is adjustable, where the processing unit is configured to determine a measurement of the force applied to at least the front component and to display the measurement on the display. In some embodiments, the one or more sensors are configured to detect photons.

In other embodiments, the device further includes a board comprising one or more holes, one or more sensors coupled to the processing unit, the one or more sensors configured to detect whether one or more objects are inside the one or more holes and to generate one or more electric signals in response to said detection; and one or more openings configured to receive the strength testing unit.

In other embodiments, the device further includes a timing unit coupled to the processing unit, the timing unit configured to measure the time elapsed between at least two electric signals generated by the one or more sensors.

In some embodiments, the device further includes a data storage unit coupled to the processing unit, the data storage unit configured to store at least the one or more corresponding force measurements. In some embodiments, the processing unit is configured to transmit the one or more force measurements to a receiving unit at remote location.

In accordance with another aspect of the present disclosure, there is provided a method comprising the steps of providing a device with a strength testing unit. The strength testing unit comprising a force sensor, a front handle configured to receive a force sensor, a first fastener; a second fastener, wherein the first and second fasteners are coupled to the force sensor; a back handle coupled to the first and second fasteners and positioned at a distance away from the front handle; a force transfer member coupled to the back handle. The method further includes the steps of allowing a user to apply force to at least the back handle, said force being transferred from the back handle to the force sensor via the first and second fastener; generating one or more electric signals corresponding to the force applied, said one or more signals generated by the force sensor; determining a measurement of the force applied, said measurement determined by a processing unit coupled to the first force sensor; and displaying the measurement on a display coupled to the processing unit.

In some embodiments, the method further includes the steps of detecting whether one or more objects are inside one or more holes on a board, said detecting is achieved by one or more sensors coupled to the one or more holes; generating one or more electric signals in response to said detection, said generating is achieved by the one or more sensors; and receiving the one or more signals by the processing unit, said processing unit is coupled to the one or more sensors.

In some embodiments, the method further includes the step of providing a time stamp to indicate when the one or more objects are inserted or removed from the one or more holes.

In some embodiments, the method further includes the step of determining by the processing unit a type of test being administered based, at least in part, on the type of force being applied to the force sensor.

In some embodiments the method further includes the steps of restraining the user's hand to isolate the portion of the hand to be tested; and connecting the isolated portion of the hand to the force transfer member; where the force applied by the user is a pulling force on the back handle. In some embodiments, the user applies the force by pushing the back handle inward toward the front handle.

In some embodiments, the method further includes the step of providing to the user information associated with the applied force, said information comprising an effective range of force to allow the user to maximize the force being applied to the device within said range.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 illustrates a schematic diagram of the strength and dexterity testing module in accordance with the present disclosure.

FIG. 2 illustrates a schematic diagram of a strength and dexterity system in accordance with the present disclosure.

FIG. 3 illustrates a schematic diagram of a strength and dexterity system populated with pegs used for dexterity testing in accordance with the present disclosure.

FIG. 4 illustrates a top view of a Grooved Peg Test in a strength and dexterity system used for dexterity testing in accordance with the present disclosure.

FIG. 5A illustrates a top view of a peg hole design to accommodate numerous dexterity tests in a strength and dexterity system in accordance with the present disclosure.

FIG. 5B illustrates a cross sectional view of peg holes designed to accommodate numerous dexterity tests in a strength and dexterity system in accordance with the present disclosure

FIG. 6 illustrates an additional attachment for a strength and dexterity testing module for larger manual muscle testing in accordance with the present disclosure.

FIG. 7 illustrates a perspective view of an alternative embodiment of the strength and dexterity system in accordance with the present disclosure.

FIG. 8 illustrates a perspective view of an alternative embodiment of the strength and dexterity testing module in accordance with the present disclosure.

FIG. 9 illustrates a perspective view of a patient's hand performing a grip strength test with one embodiment of the strength and dexterity testing module in accordance with the present disclosure.

FIG. 10 illustrates a perspective view of a patient's hand performing a grip strength test with one embodiment of the strength and dexterity testing module in accordance with the present disclosure.

FIG. 11 illustrates a perspective view of a patient's hand performing a pinch test with the strength and dexterity testing module in accordance with the present disclosure.

FIG. 12 illustrates a demonstration of intrinsic hand muscle testing of a hypothenar muscle with peg restraints in accordance with the present disclosure.

FIG. 13 illustrates demonstration of intrinsic hand muscle testing of a first dorsal interosseous muscle with peg restraints in accordance with the present disclosure.

FIG. 14 illustrates demonstration of intrinsic hand muscle testing of an abductor pollicis brevis muscle with peg restraints in accordance with the present disclosure.

FIG. 15 illustrates demonstration of intrinsic hand muscle testing of an opponens pollicis muscle with peg restraints in accordance with the present disclosure.

FIG. 16 illustrates a flow diagram for testing strength and dexterity in accordance with the present disclosure.

FIG. 17 illustrates a flow diagram for processing signals obtained from strength and dexterity tests in accordance with the present disclosure.

FIG. 18 illustrates an example of a force diagram generated by a load sensor from the strength and dexterity tests in accordance with the present disclosure.

FIG. 19 illustrates a flow diagram of an automated method for measuring dexterity in accordance with the present disclosure.

FIG. 20 illustrates a time versus signal graph generated by the strength and dexterity system in accordance with the present disclosure.

FIG. 21 illustrates a schematic diagram of a microprocessor system in accordance with the present disclosure.

FIG. 22 illustrates a flow diagram of a method for performing a strength and dexterity test using a Theraband® in accordance with the present disclosure.

FIG. 23 illustrates an example of a user interface for rehabilitative testing in accordance with the present disclosure.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular embodiments illustrated herein.

### DETAILED DESCRIPTION

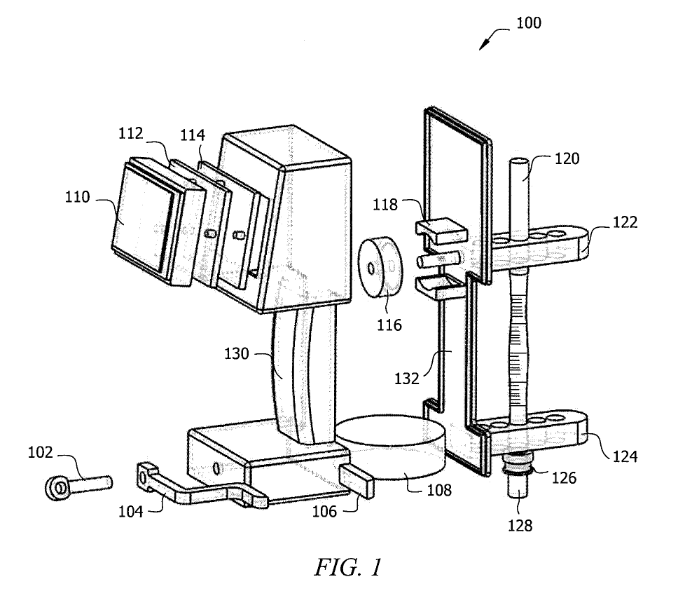

In general, one or more implementations described herein are directed to a device/system for testing muscle strength and dexterity in the hand and upper extremities. Various implementations of the device/system for testing muscle strength and dexterity in the hand and upper extremities will be described in more detail with reference to FIGS. 1-23.

FIG. 1 illustrates a schematic diagram of a strength and dexterity testing module in which the various techniques described herein may be incorporated and practiced. The schematic diagram illustrates an exploded view of the strength and dexterity testing module 100. The strength and dexterity testing module 100 may include an eyebolt 102, a first component 104 of the pinch tester, a second component 106 of the pinch tester, a first load or force sensor 108, a display 110, a microprocessor/circuit board 112, a battery source 114, a second load or force sensor 116, a load sensor holder 118, a back handle 120, a first fastener 122, a second fastener 124, a lock collar 126, a threaded screw 128, a front handle 130 and a back plate 132. Although the strength and dexterity testing module 100 is described as having these components, it should be noted that in other implementations the strength and dexterity testing module 100 may include additional components or may not include all of the components listed above. The following description of the strength and dexterity testing module 100 is provided to serve as an example of the strength and dexterity testing module 100 and is not intended to limit the scope of the claims.

Referring to FIG. 1, in one implementation, the strength and dexterity testing module 100 may be assembled such that the eyebolt 102 may be coupled to the strength and dexterity testing module 100 via the first load sensor 108. The first load sensor 108 may be loaded in tension or compression and placed at the bottom of the strength and dexterity testing module 100. In one implementation, the eyebolt 102 may screw through the front panel of the module 100 and into the first load sensor 108. In this manner, pulling the eyebolt 102 may create a tension force between the eyebolt 102 and the first load sensor 108. The eyebolt 102 may be pulled via a loop (not shown) made of nylon, Velcro or any other similar non-elastic material. The loop may be secured to the eyebolt 102 via the eye of the eyebolt 102. The eyebolt 102 may also be coupled to the first component 104 of the pinch tester. The first component 104 of the pinch tester may be a piece of metal having a thread such that the eyebolt 102 may screw into the thread.

Referring to FIG. 1, the second component 106 of the pinch tester may be a metal piece that extends from the side of the strength and dexterity testing module 100. The distance between the first component 104 and the second component 106 may be adjusted by rotating the first component 104 about the eyebolt 102. The second component 106 may remain rigid and attached to the strength and dexterity testing module 100 such that it is immobile with respect to the strength and dexterity testing module 100. The position of first load sensor 108 may be close to the bottom of the strength and dexterity testing module 100 to eliminate off-axis loading intrinsic hand muscle testing and to allow the loop to fit around a patient's finger in a perpendicular fashion. In this position, the first load sensor 108 may also be configured to facilitate both the pinch and the intrinsic hand muscle testing. Although the strength and dexterity testing module 100 has been described as having an eyebolt 102, it should be noted that the eyebolt 102 may be replaced with any object that can be attached to a loop or other force transfer components that may be used to test the muscle strength of a muscle in a hand. Alternatively, other embodiments do not include the eyebolt, and the loop or other force transfer components are directly attached to the load or force sensor. The force transfer components suitable for use in the present disclosure may include anything that is capable of transferring the force generated by a subject to a force or load sensor.

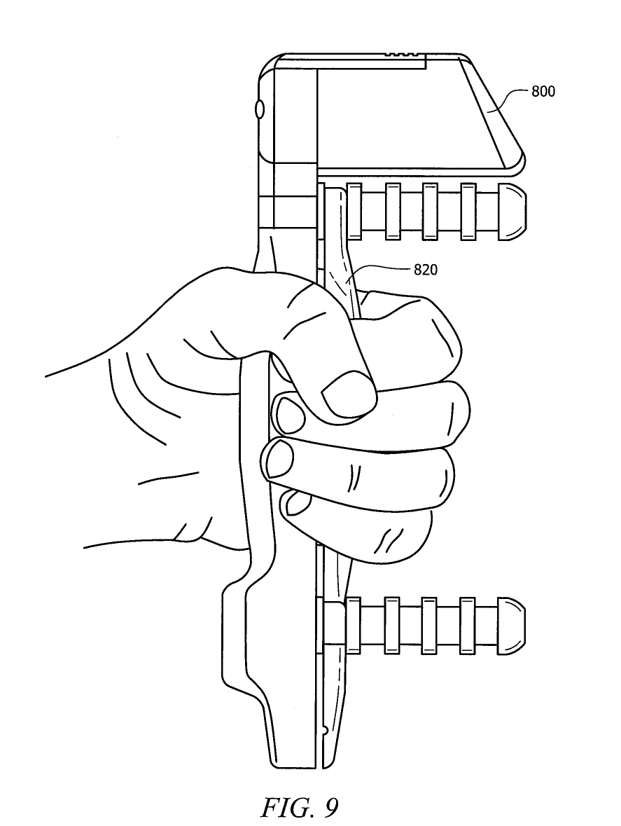

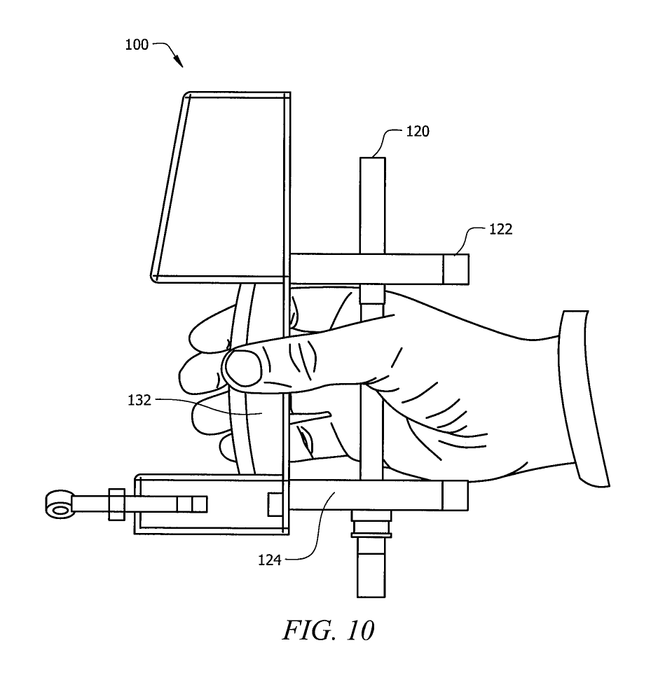

Referring to FIG. 1, in operation, a patient (e.g., user) may pull a loop (not shown) secured through the eyebolt 102 with one or more fingers to test his intrinsic hand muscle strength. This is further described in details later with reference to FIGS. 9 and 10. In response to the pulling of the loop, the first load sensor 108 may generate an electric output (voltage) that corresponds to the force exerted by the patient pulling the loop. In one implementation, the electric output may be an analog signal. The first load sensor 108 may be coupled to the microprocessor/circuit board 112 such that the analog signal may travel directly to the microprocessor/circuit board 112. The signal from the first load sensor 108 represents the force exerted by the patient.

In one implementation, the microprocessor/circuit board 112 may include a microprocessor and one or more circuit components such as filters, amplifiers and the like. Although in this implementation, the microprocessor and other circuit components are described as being located on the same circuit board 112, it should be noted that in other implementations the microprocessor and the circuit board may be separated. The circuit in the microprocessor/circuit board 112 may filter noise and amplify a signal received from the first load sensor 108 or 116. In one implementation, the microprocessor on the microprocessor/circuit board 112 may be used to control the signal sampling rate and to display the forces indicated by the first load sensor 108 on the display 110. The microprocessor/circuit board 112 may display the force exerted by the patient on the first load sensor 108, one or more force diagrams and a timer that may be used for dexterity testing. In one implementation, the microprocessor/circuit board 112 may be coupled to the display 110. The display 110 may be a touch screen, LCD or any other type of video display device that is capable of displaying graphics from the microprocessor/circuit board 112. In another implementation, the display 110 may be a separate attachment from the strength and dexterity testing module 100 such that it may swivel about the vertical axis through a threaded bolt (or through another mechanism) on the strength and dexterity testing module 100. This feature may provide the clinician more flexibility in providing the patient a view of the display 110.

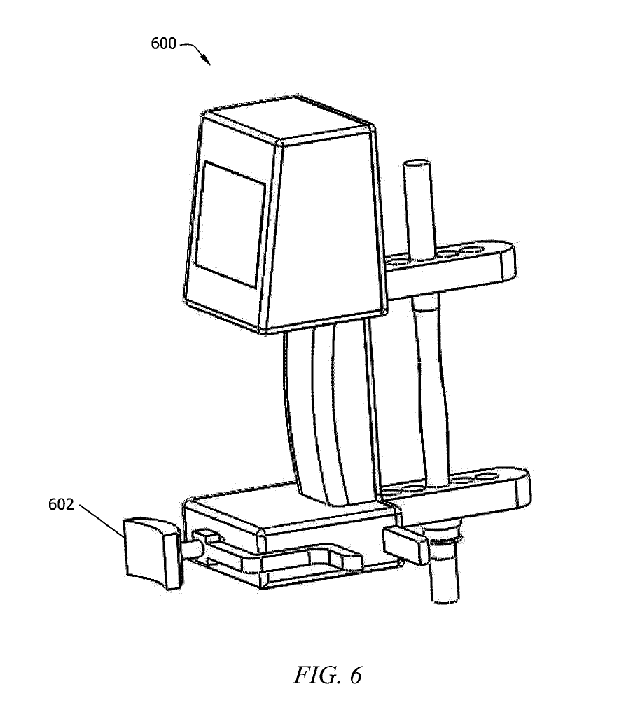

Referring to FIG. 1, the second load sensor 116 may be located above the first load sensor 108, toward the top of the testing module 100. As shown, the second load sensor 116 may include a circular hole that passes through the body of the second load sensor 116 in the axial direction such that it may sense or measure a shear strain or a simple compression. In one implementation, the shear strain loading may occur when the second load sensor 116 is placed perpendicularly with the axis of force, i.e., the circular hole is parallel to the direction of force applied by the user. For instance, the cylindrical extension of first fastener 124 extends through the back plate 132 and fits through the circular hole in the second load sensor 116. Referring to FIGS. 1 and 6, when grip is applied, the cylindrical extension of the first fastener 124 generates a force towards the front. This force is sensed as shear loading by the second load sensor 116. The second load sensor 116 may be positioned close to where a patient's thumb may apply a force to the back handle 120 when performing the grip test. This position allows for a more direct assessment of grip strength. The second load sensor 116 may be fastened to the holder 118, bolted to the top of the holder 118 or bolted through the back plate 132.

Referring to FIG. 1, when a patient pushes along the surface of the front handle 130, the second load sensor 116 may compress such that it may sense the shear force being applied to it. Here, the second load sensor 116 may be placed against a rigid surface and determine the force pushing back against the patient's force. In another implementation, the force exerted by the patient may be applied directly to the second load sensor 116 in simple compression. For instance, simple compression occurs when the second load sensor 116 acts as a solid rigid face, i.e., without any circular hole at the center, that senses force applied directly to that face. In one implementation, the patient may place his thumb on the back handle 120 and his other fingers on the front handle 130 as shown in FIG. 6. As the patient increases his grip between the back handle 120 and the front handle 130, the second load sensor 116 may compress such that it produces an analog signal (e.g., voltage) proportional with the increasing grip strength. The front handle 130 may include fitted depressions such that the patient's fingers may fit within designated spaces on the strength and dexterity testing module 100. Again, the second load sensor 116 may be coupled to the microprocessor/circuit board 112. The circuit in the microprocessor/circuit board 112 may operate to filter and amplify the analog signal received from the second load sensor 116 as described above for the first load sensor 108. The load sensors 108 and 116 may be positioned in order to allow the pinch, grip and intrinsic hand muscle testing to coexist in one system.

Referring to FIG. 1, the battery source 114 may provide power to the first load sensor 108, the second load sensor 116, the display 110, and the microprocessor/circuit board 112. In one implementation, the battery source 114 may be a lithium ion battery, but in other implementations the battery source 114 may be replaced by a continuous source of electrical energy. The microprocessor/circuit board 112 may include a communication module (not shown) that may transmit one or more analog signals obtained from the first load sensor 108 or the second load sensor 116 to a secure backend computer or server. In one implementation, the communication module of strength and dexterity testing module 100 may include wired communication means such as an Ethernet interface to communicate with various networks, e.g., LAN, and a telephonic interface to transmit and receive data via the public switched telephone network (PSTN), and/or wired communication means such as wireless network adapters and/or Bluetooth to communicate with wireless networks, e.g., Wi-Fi, Bluetooth, and hardware to communicate with wireless telecommunication networks, e.g., 3 G and 4 G. It should be noted that the communication module is not limited to these communication means. In one implementation, the microprocessor/circuit board 112 may filter the communication signals with active low pass filtering techniques and the like. In another implementation, the microprocessor/circuit board 112 may also amplify the communication signals with instrumentation amplifiers and the like.

The back handle 120 may be designed such that the strength and dexterity testing module 100 may rotate 360° about the vertical axis of the back handle 120. The ability of the back handle 120 to rotate may allow the device to be self-correcting and may also eliminate any off-axis loading issues that may arise when the patient pulls against the loop attached to the eyebolt. Furthermore, free rotation may allow the testing of a wide range of hand sizes and irregular morphologies. In one implementation, the back handle 120 may be a cylindrical rod. The back handle 120 may be fitted through the first fastener 124 and the second fastener 124 that extend out from the back of the strength and dexterity testing module 100. In one implementation, the back handle 120 can be repositioned and fitted in a number of through holes in the fasteners 122 and 124. Each of the through holes may represent a different position for the back handle 120 that may be used for grip testing. In one implementation, each of these positions may conform to established clinical guidelines and standardized protocols endorsed by the American Society of Hand Therapists. The fasteners 122 and 124 may also include length markings (e.g., metric and/or British) to record positioning for repeated testing. In one implementation, the fasteners 122 and 124 may operate like a Woodruff key. Generally, a Woodruff key, as with other mechanical keys, prevents relative rotation between the two parts and allows torque to be transmitted through.

The back handle 120 may also include a lock collar 126. The lock collar 126 may translate in the vertical plane along the axis of the back handle 120 such that the back handle 120 may slide up or down through the fasteners 122 and 124 and be removed from one set of holes and inserted in another set of holes in the fasteners 122 and 124. The lock collar 126 may then lock the back handle 120 in the corresponding holes of the fasteners 122 and 124. In one implementation, the lock collar 126 may rotate within the fasteners 122 and 124. The portion of the back handle 120 between the fasteners 122 and 124 may include a foam covering.

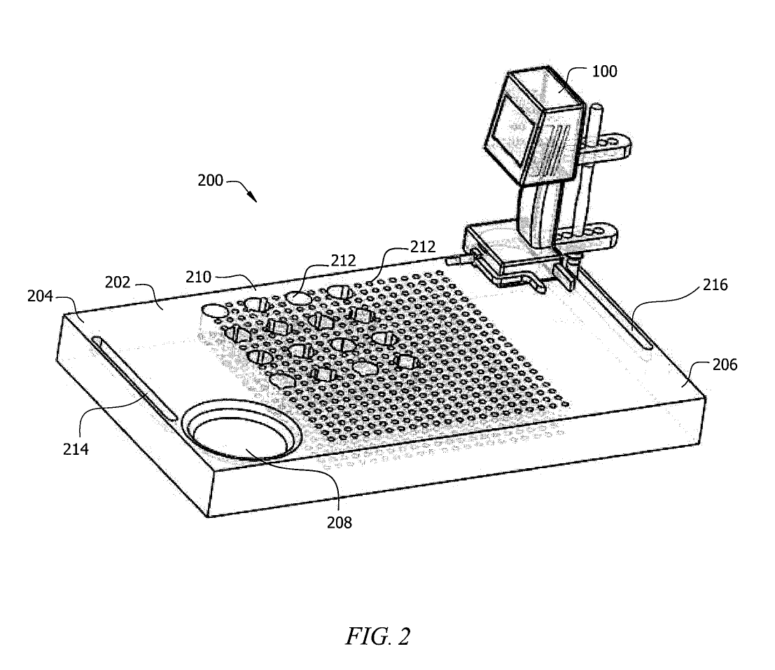

In another implementation, the back handle 120 may attach the strength and dexterity testing module 100 to the strength and dexterity system 200. The strength and dexterity system 200 will be described in more detail in FIG. 2. The back handle 120 may also be used as a bar that an individual (e.g., user) grips to generate grip strength in the grip test. A second back handle (not shown) may be added to the strength and dexterity testing module 100 directly behind the back handle 120 to create a more ergonomic interface for grip strength. The second back handle may be bolted to the testing enclosure 100 at a distance from the back handle 120 such that it accommodates additional (e.g., larger) hand sizes. The second back handle may also be coupled to the fasteners 122 and 124. In a strength and dexterity testing module 100 having two back handles, the second back handle may be used to couple the strength and dexterity testing module 100 with the strength and dexterity system 200. In implementations having two back handles, the fasteners may have extended Woodruff keys with two through holes for each back handle.

In yet another implementation, a load sensor may be implanted directly into the back handle 120. This may be used to replace or in parallel with the second load sensor 116. The load sensor implanted into the back handle 120 may act as a multi-axis load sensor that is in-line with back handle 120. The load sensor implanted into the back handle 120 may also act as a strain gauge that may be attached to the exterior of the back handle 120. Here, the load sensor may be built directly into the back handle 120 such that it will be cylindrical and the same width as the back handle 120. The load sensor may also have an additional extension that may sense how much the back handle 120 may bend when a grip is applied.

Furthermore, the front handle 130 can be used by a clinician to measure intrinsic hand muscle strength through first load sensor 108 for patients with abnormal hand morphologies or limitations in joint motion. In this manner, the patient may push against the front handle 130 and the second load sensor 116 may detect the force applied by the user. In one implementation, the pinch and grip testing may also be performed in this manner as well.

FIG. 2 illustrates a schematic diagram of a strength and dexterity system 200 in accordance with one or more implementations of various techniques described herein. In one implementation, the strength and dexterity system 200 may include a pegboard 202, a first side 204 of the pegboard 202, a second side 206 of the pegboard 202, a circular depression 208, a built-in dexterity tester 210, one or more peg holes 212, a first slot 214, and a second slot 216. Although the strength and dexterity system 200 is described as having the above listed components, it should be noted that in other implementations the strength and dexterity system 200 may include additional components or may not include all of the components listed above. As such, the following description of the strength and dexterity system 200 is provided to serve as an example of one implementation of the strength and dexterity system 200 and is not meant to limit the scope of the claims.

Referring to FIG. 2, in one implementation, the strength and dexterity testing module 100 may be integrated within a strength and dexterity system 200 such that the strength and dexterity testing module 100 may fit into one or more slots 214 or 216 located on either side of the pegboard 202 (e.g., first side 204, second side 206). As shown by FIG. 2, testing module 100 is attached to pegboard 202 at slot 216. Referring to FIGS. 1 and 2, one way module 100 may be integrated to pegboard 202 is that the bottom of the back handle 120 may flush into a threaded screw 128, which is designed to interface within either fitted slots 214 or 216 on either side of the strength and dexterity system 200. The fitted slots 214 and 216 on either side of the pegboard 202 may allow the strength and dexterity testing module 100 to couple to the pegboard 202 via a threaded screw or the like. The strength and dexterity testing module 100 can translate or move along these slots via ball bearings and other similar devices. Additionally, the strength and dexterity testing module 100 can be removed from the slot such that it may be used as a handheld device or it can be moved to the opposite side of the strength and dexterity system 200 to accommodate additional testing. In one implementation, the slot in which the strength and dexterity testing module 100 may fit into the pegboard 202 may also serve as a charging station for the testing module 100 via an AC powered or USB-linked cable.

Referring to FIG. 2, the strength and dexterity system 200 may be composed of peg holes 212. Peg holes 212 may be dispersed throughout the pegboard 202 and placed at strategic locations on the pegboard 202 such that pegs or other objects may be placed inside the peg holes 212. The pegs may be used for various dexterity tests. In one implementation, the locations of pegs on the pegboard 202 that were used for a particular patient and a specified test may be recorded by the microprocessor/circuit board 112. As such, a clinician may use the recorded peg locations to repeat the specified test for the particular patient. By placing the pegs in a similar manner as indicated by the recorded peg locations, the clinician may be able to consistently obtain data under identical conditions.

The peg holes 212 are dispersed throughout the pegboard 202 to accommodate various hand sizes and hand morphologies and also to test different intrinsic hand muscles of a patient. The pegs may also be used to restrain a patient's hand such that only the muscle of interest is tested. Preferably, the holes for restraining the hand in IHMS measurement are hexagonal, which is optimized to ensure hand and wrist restraint. In one implementation, the strength and dexterity testing module 100 may be used without pegs. Here, the patient may place his hand on the pegboard and the loop is fitted around the muscle of interest. Without having a peg to restraint his movement, the patient can pull against the loop and generate a force that may be displayed by the microprocessor/circuit board 112.

The built-in dexterity tester 210 may include holes with a specified diameter, depth and spacing to facilitate the use of larger pegs and to conform to clinically validated parameters that are standard for dexterity testing. In one implementation, pegs may be stored in a circular depression 208. Although the circular depression 208, the built-in dexterity tester 210 and the peg holes 212 are illustrated as being in a specific area of the pegboard 202, it should be noted that the positions of these components may be moved to any part of the pegboard 202. For example, the built-in dexterity tester may be placed on the right side, left side or centered on the pegboard 202.

In one implementation, there may be variations in the width and spacing of the peg holes 212 to facilitate various types of clinical dexterity tests. The number of peg holes 212 for the built-in dexterity test may also be altered to facilitate additional types of clinical dexterity tests. Further, the organization of the peg holes 212 for the built-in dexterity test can be varied from an evenly spaced rectangular format, to a staggered fashion or any other geometry. The peg holes 212 may also be instrumented with LEDs or sensors that may be coupled to the module 100. In one implementation, the positions of the peg holes 212 that contain pegs for a specific test may be identified and saved by the microprocessor/circuit board 112. Each time a patient is to perform a specific test, the microprocessor/circuit board 112 may then display the peg holes 212 that are to have pegs for the corresponding test based on the patient's previous test information. By storing information about the testing conditions, e.g., positions of the peg holes 212 having pegs for each patient, the clinician may minimize variations between the patient's subsequent tests. The peg holes 212 may be marked by gridlines or given a distinct positional marking (e.g., A1, A2, A3, etc.) such that peg holes 212 are easily identified and to ensure repeatable testing conditions. In one implementation, the pegs may be shaped as a cylinder such that the pegs would fit within the peg hole 212. Pegs may also be used to position or restrain a patient's arm/hand during a test. Additionally, pegs may be used to accommodate different hand morphologies on the strength and dexterity system 200. For instance, pegs may include U shaped cups and/or bars that can support a hand and a forearm. Pegs may also include straps to wrap around the patient's digits, hand, wrist or forearm.

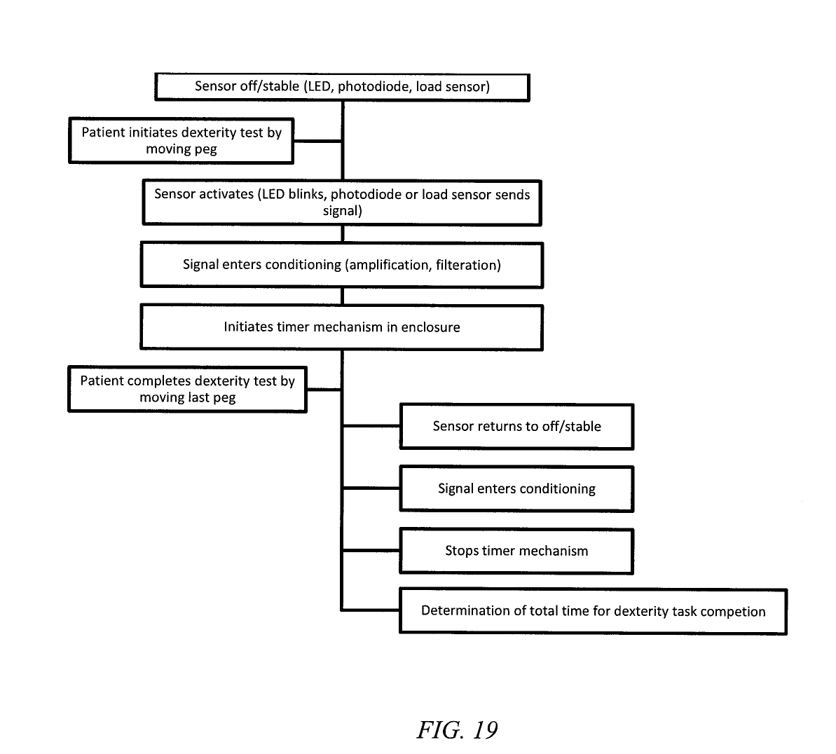

In one implementation, the pegboard 202 may include photodiodes within each peg hole 212 and each peg may include an LED on its end. The LED on the end of the peg may provide a light signal to the photodiode when it is inside the peg hole 212. In another implementation, the peg hole 212 may include an LED and a photodiode and each peg may include some reflective material on its end. Here, the LED and the photodiode may act in a reflective mode such that the photodiode may react and sense the wavelength emitted by a specific LED. Here, the signal sent from the LED may be directly sensed by the photodiode due to a reflection from a side of the pegboard or the peg itself. Although the peg hole and the peg have been described as having an LED and a photodiode, it should be noted that in other implementations the peg hole and peg may include some other technology that may allow the microprocessor/circuit board 112 to determine whether the peg is inside the peg hole 212. One way the sensors can communicate with the microprocessor/circuit board 112 is through wireless communications such as Bluetooth or the sensors can be connected via wires to a port, e.g., USB port, located on the pegboard 202 that connects to testing module 100 when the testing module is connected to the pegboard 202 at slots 214 or 216. By determining whether the peg is inside the peg hole, the microprocessor/circuit board 112 can determine when the peg was placed or removed from the peg hole which can be used for dexterity testing. A method for using the microprocessor/circuit board 112 in dexterity testing is described in more detail with reference to FIG. 19.

Any intrinsic muscle can be tested by changing the position of the hand on the pegboard 202 and appropriately placing the loop around the finger. Generally, a patient may be instructed to place his hand on the pegboard 202 such that the muscle of interest can be tested. After the patient's hand is placed in a specified manner on the pegboard 202, a clinician may place pegs on the pegboard to indicate the manner in which the patient's hand is placed on the pegboard 202 and to restrain the patient's hand such that the muscle of interest is isolated for testing. In this manner, the pegs may restrain the patient's muscles except for the patient's muscle of interest. A loop may then be placed such that the patient may pull against the loop using the muscle of interest. The loop may be coupled to the eyebolt 102 which is coupled to the first load sensor 108. The first load sensor 108 may detect the force exerted by the patient's muscle of interest, and the microprocessor/circuit board 112 may analyze or store the force data received from the first load sensor 108. In one implementation, the pegs may be deemed unnecessary and the muscle of interest may be tested using just the eyebolt 102 coupled to first load sensor 108 without the pegboard 202 or without the pegs. Examples of specific intrinsic muscle tests are described in detail below. Each of the examples described below are presented for illustrative purposes and are not meant to limit the scope of the invention.

Although the strength and dexterity system 200 has been described as having multiple components stored therein, it should be noted that each of the components on the strength and dexterity testing module 100 and the strength and dexterity system 200 may also be used as separate devices. For example, the strength and dexterity testing module 100 may be used separately without the pegboard 202. In this implementation, the strength and dexterity testing module 100 may be used to test pinch strength components, grip strength components and intrinsic hand strength components without having the complete strength and dexterity system 200. In this example, the strength and dexterity testing module 100 may be used as a handheld device. In another implementation, the strength and dexterity testing module 100 may only include a pinch strength tester and an intrinsic hand strength tester without the grip strength tester. In yet another implementation, the built-in dexterity test board 210 may be used without load sensors for grip strength, pinch strength and intrinsic hand strength tests.

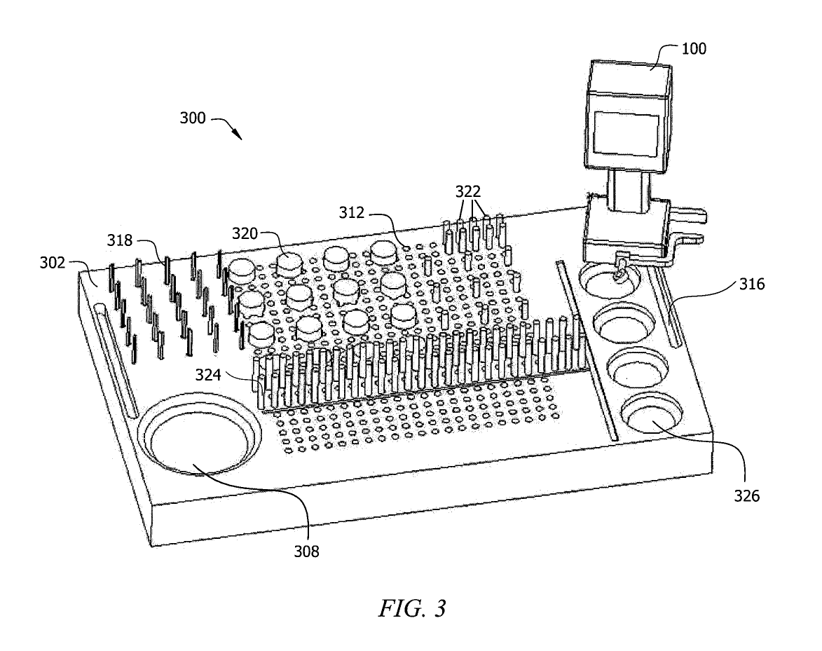

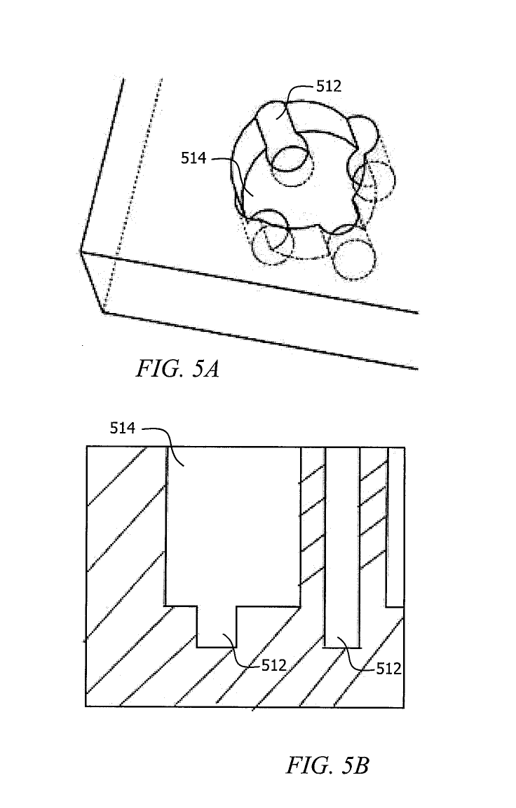

FIG. 3 illustrates a schematic diagram of a strength and dexterity system 300 populated with pegs used for dexterity testing in accordance with one or more implementations of various techniques described herein. In one implementation, the strength and dexterity system 300 may include several dexterity tests including the Grooved Peg Test 318, the Functional Dexterity Test 320, pegs 322 used for intrinsic hand muscle testing, the Purdue Pegboard Test 324 and corresponding circular depressions 326 for storing Purdue-specific pegs 324. A circular depression 308 may be used to store pegs 322. FIG. 3 provides an example of how different dexterity tests may be implemented on the pegboard 302. However, it should be noted that the peg holes 312 may be created such that various dexterity tests can be performed on the pegboard 302. One example of how the peg holes 312 may be constructed is described in FIG. 5 below.

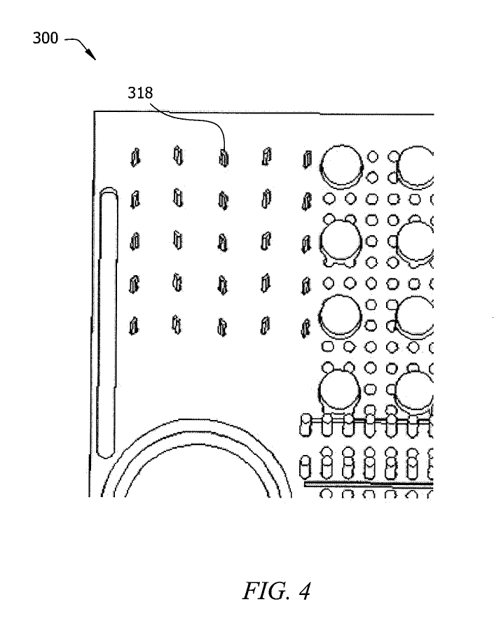

FIG. 4 illustrates a top view of a Grooved Peg Test 418 that can be used for dexterity testing in various implementations of a strength and dexterity system described in the present disclosure, such as systems 200 and 300. In generally, the Grooved Peg Test 418 is a manipulative dexterity test. In one implementation, the Grooved Peg Test 418 incorporates holes that are irregularly shaped and randomly organized. The shape of the pegs of the Grooved Peg Test 418 may be configured to match the irregular shape of the holes, which requires a patient to rotate the pegs to match the hole before the pegs can be inserted in the holes.

FIG. 5A illustrates a top view of a peg hole designed to accommodate numerous dexterity tests in a strength and dexterity system in accordance with one or more implementations of various techniques described herein. In one implementation, the peg holes may be drilled throughout the pegboard in a variety of depths and diameters such that various pegs may fit into the same pegboard, e.g., board 202 or 302. In another implementation, a hole configured to receive thinner pegs can be integrated with a hole configured to receive thicker dexterity pegs. For instance, referring to FIG. 5A, the peg holes 512 may be deeper than the peg dexterity holes 514. This allows the peg holes 512 and peg dexterity holes 514 to integrate together. Referring to FIG. 5B, the cross sectional view of such an integrated hole with the peg hole 512 being deeper than the dexterity hole 514. FIG. 5B also shows that certain peg holes are drilled at different depths to enable multiple dexterity tests to be integrated within one system. Pegs of varying sizes can be used to fit within the specific peg dexterity hole depending on which dexterity test is being used or whether restraint pegs are being used for intrinsic hand muscle strength testing. The peg holes may be constructed to fit pegs of any width or size in order to facilitate various dexterity tests on the same pegboard.

FIG. 6 illustrates an additional attachment for a strength and dexterity testing module 600 for larger manual muscle testing in accordance with one or more implementations of various techniques described herein. In one implementation, the additional attachment 602 may include a flat plate or semi-curved plate instead of the eyebolt, e.g., eyebolt 102 shown in FIG. 1. The additional attachment 602 may be used for manual muscle testing of other muscles such as muscles related to the shoulder, forearm, knee, etc. The flat plate attachment may also be replaced with a larger handle bar to test wrist strength. For this variation, the fingers can wrap around the handle bar and the strength and dexterity testing module 100 may be positioned further up the vertical axis and locked via the lock collar, e.g., lock collar 126 in FIG. 1. The additional attachment 602 may also be in the shape of a U or a bar to accommodate unrestrained muscle testing for larger muscles.

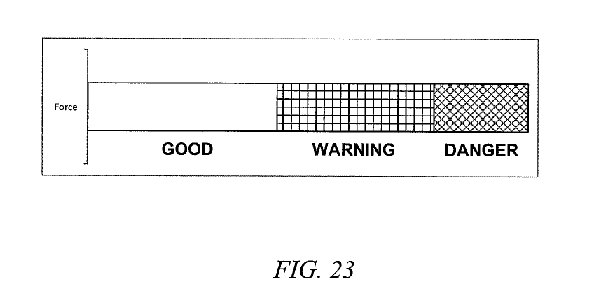

In one implementation, the additional attachment 602 may be replaced by an elastic material that may allow for a joint range of motion. The elastic material may be looped around the circular opening of the eyebolt 102 of FIG. 1 as an additional component. This elastic material may be made of, but not limited to, Therabands® and/or rubber bands of differing widths and resistances. The Therabands® may be placed around the patient's finger of interest. The patient may then be instructed to pull against the Theraband® in a particular motion dictated by the clinical protocol selected by the clinician. This is a standard method of increasing muscle strength through resistance training. However, the Theraband® may provide resistance as it deforms proportionally to the force being generated by the patient's finger. The force reading of the first load sensor 108 may be presented on the display. In one implementation, the display may provide a real-time bio-feedback to the patient as shown in FIG. 23. The real-time bio-feedback may allow the patient to maximize his efforts within an effective force range and prevent over-exertion. The bio-feedback may reduce the likelihood of damaging a muscle/tendon unit being rehabilitated. Additionally, the display can aid the patient by displaying proper technique for a given exercise, number of repetitions performed, number of repetitions remaining, elapsed time, maximum force and the like.

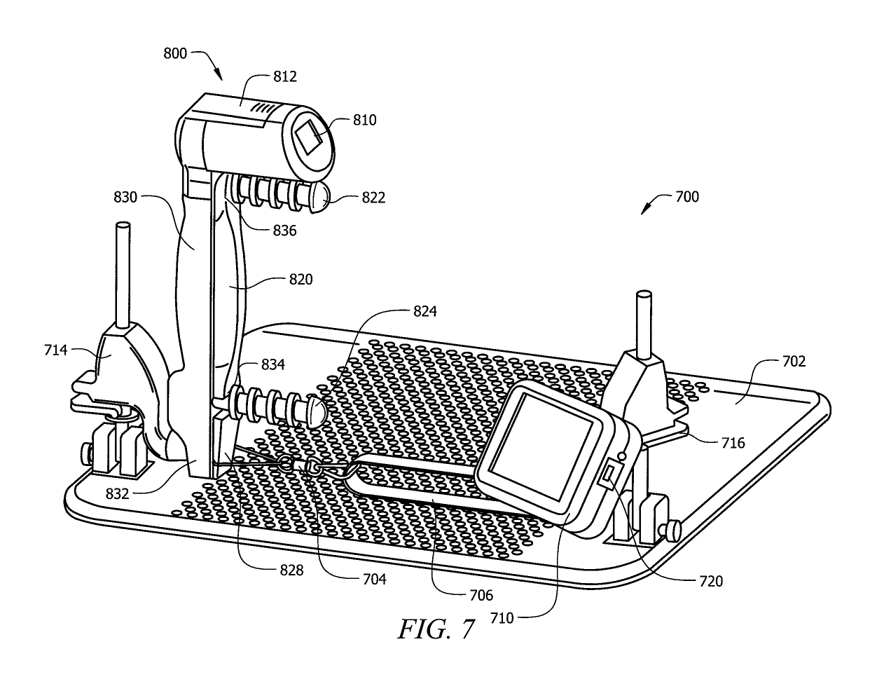

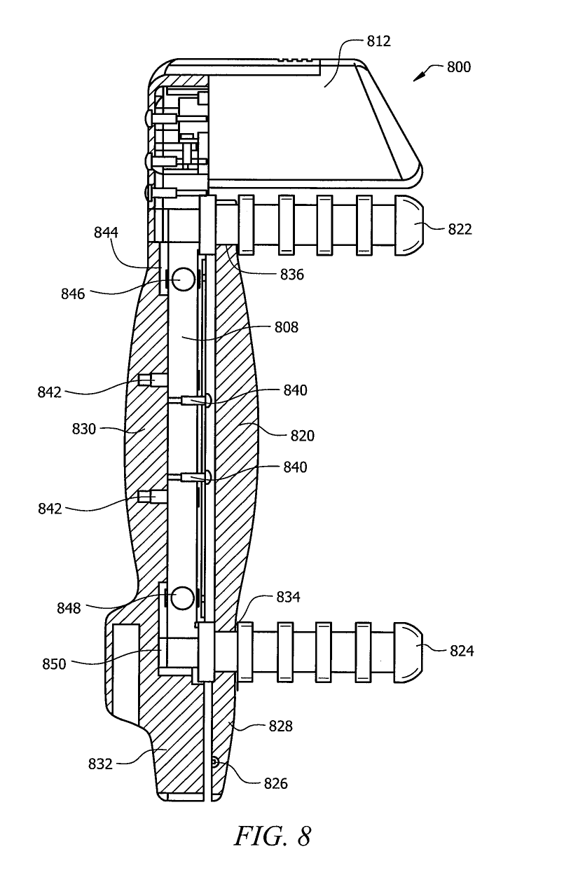

FIG. 7 illustrates an alternative embodiment of the present disclosure. In FIG. 7, the strength and dexterity system 700 may include a strength and dexterity testing module 800, a pegboard 702, a force or load transfer member 704, a strap 706, a display panel 710, and device holders 714 and 716. FIG. 8 provides a partial cross-sectional view of the testing module 800. Referring to FIGS. 7 and 8, testing module 800 may include a load or force sensor 808, a display 810, a microprocessor/circuit board (not shown) located within the display compartment 812, a back handle 820, a front handle 830, a first fastener 822, and a second fastener 824. Similar to testing module 100, testing module 800 also includes a microprocessor/circuit board and an energy source, such as a battery, which are not shown. The microprocessor/circuit board 112 described previously may be used in the testing module 800. Accordingly, the descriptions of the microprocessor/circuit board 112 apply equally to the microprocessor/circuit board of the testing module 800. Although the strength and dexterity system 700 and testing module 800 have been described as having these components, it should be noted that in other implementations may include additional components or may not include all of the components listed above. The following description of the strength and dexterity system 700 and testing module 800 is provided to serve as an example of one embodiment of the present disclosure and is not intended to limit the scope of the claims.

Referring to FIGS. 7 and 8, in one implementation, the strength and dexterity testing module 800 may be assembled such that the load sensor 808 sits inside the front handle 830. Preferably, the load sensor 808 is attached to the front handle 830 by screws 840 and 842. The body of the load sensor 808 fits snugly in the front handle 830. The front handle 830, however, has a clearance 844 near the top and a clearance 850 near the bottom to accommodate any movements by the top and bottom ends, respectively, of the load sensor 808. The snug fit and distance between the ends of the load sensor 808 isolates the movements of the ends from one another. That is, if only the top end of the load sensor 808 is pushed inward or deflected, that force does not affect or displace the bottom end of the load sensor 808. In the preferred embodiment, load sensor 808 also includes a top sensor 846 and a bottom sensor 848. Any displacement or deflection of the top or bottom sensor 846 or 848 in the inward direction toward the front handle 830 or outward direction toward the back handle 820 generates an electrical signal (voltage). This can be achieved by means known to one skilled in the art. One such means is providing a voltage across the circular cutouts of the top or bottom sensor 846 or 848 and measuring the voltage drop when either the top or bottom sensor 846 or 848 is deflected.

As discussed above, the movements by one end of the load sensor 808 does not affect the voltage measurement of the other end. One advantage provided by the present disclosure is that the load sensor 808 can determine the type of test being administered based solely on the force being applied. For instance, in a grip test, both ends of the load sensor 808 will be deflected, resulting in an electrical signal being generated by both ends to inform the microprocessor that a grip test is being performed. In a pinch test, only the bottom end 848 of the load sensor 808 is deflected inward, resulting in an electrical signal being generated only by the bottom end 848. In a IHMS measurement or test, only the bottom end 848 of the load sensor 808 is deflected outward, resulting in an electrical signal being generated by the bottom end 848. As the pinch test and IHMS measurement involve inward and outward deflection, respectively, the electrical signal generated can indicate the different angle of deflection and inform the microprocessor of the type of test being performed. Consequently, it is not necessary for a user to select the type of test to be administered as the device is designed to recognize the force and as a corollary, the type of test, being applied.

As shown in FIG. 8, the ends of the load sensor 808 are coupled to the first and second fasteners 822 and 824, respectively. The top of back handle 820 is also connected to the first fastener 822 at a first fastener attachment point 836 and the bottom of the back handle 820 is attached to the second fastener 824 at a second fastener attachment point 834. In this configuration, any force applied to the back handle 820 in the inward direction toward the front handle 830, e.g., grip strength, is directly translated to fasteners 822 and 824. Consequently, this force correspondingly displaces both ends of the load sensor 808 further inward toward the front handle 830.

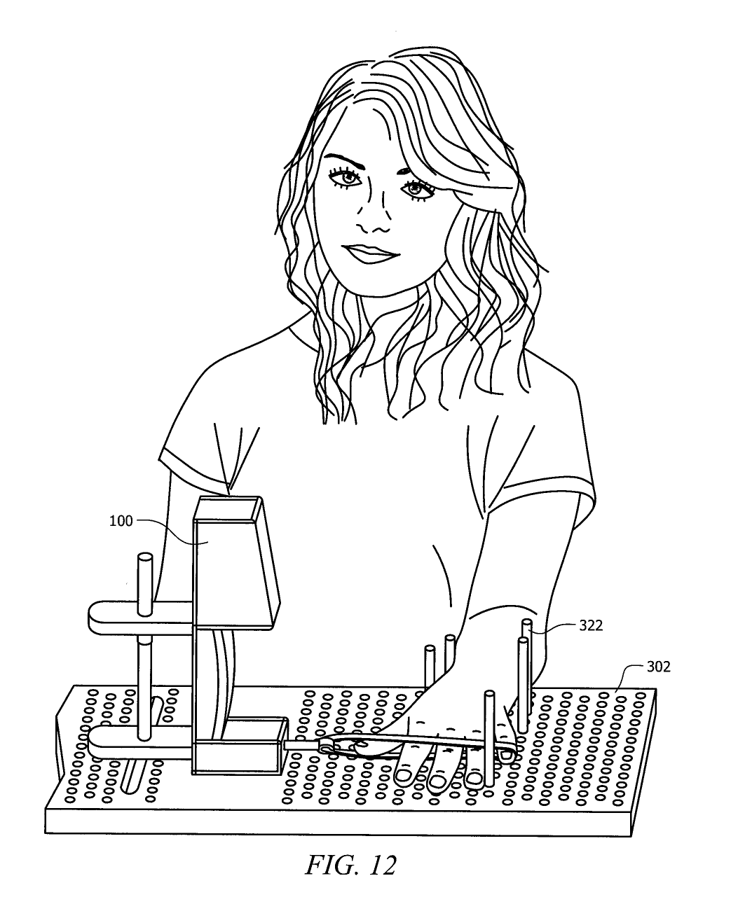

Referring to FIGS. 7 and 8, in operation, a patient (e.g., user) may pull the strap 706 with one or more fingers to test his intrinsic hand muscle strength. This is further described in details later with reference to FIGS. 12-15. In response to the pulling of the strap 706, the load sensor 808 may generate an electric output (voltage) that corresponds to the force exerted by the patient pulling the strap 706. Because the bottom of the back handle 830 is coupled to the second fastener 824, which is coupled to bottom end of the load sensor 808, any force pulling on the bottom 828 of the back handle 820 will be translated to the bottom sensor 848 via the second fastener 824. In one implementation, the electric output may be an analog signal. The load sensor 808 may be coupled to the microprocessor/circuit board in the display compartment 812 such that the analog signal may travel directly to the microprocessor/circuit board. The signal from the load sensor 808 represents the force exerted by the patient.

Referring to FIG. 7, the force transfer member 704 is preferably optimized for a complete transfer of the force applied by the intrinsic hand muscle. That is, the force transfer member 704 has a connector component 722 that allows the two ring components 722 to fully rotate independent of one another. Accordingly, both ring components 722 have complete freedom of movement. As such, any force applied during the intrinsic muscle test will be completely transferred. Otherwise, any hindrance in the movements of the ring components 722 can distort the applied force.

Referring to FIG. 8, the bottom 828 of the back handle 820 and the bottom 832 of the front handle 830 also serve as the first and second components of the pinch tester. The bottom 828 has a tapering shape to accommodate the fingers of the patients, and the bottom 832 is shaped to accommodate the thumb of a patient. The distance between the bottom 828 of the back handle 820 and the bottom 832 of the front handle 830 may be adjusted by moving the back handle 820 to the various positions on the first and second fasteners 822 and 824. When the patient performs the pinch test, such as placing the thumb on the bottom 828 of the back handle 820 and the index finger of the same hand on the bottom 832 of the front handle 830 and squeezing the thumb and finger, the load sensor 808 may generate an electric output (voltage) that corresponds to the force exerted by the patient. The signal may similarly be transmitted to the microprocessor/circuit board in the display compartment 812. As discussed above, the distance between the front handle 830 and the back handle 820 may be adjusted by moving the back handle 820 further away from the front handle 830.

Referring to FIGS. 7 and 8, one way this can be done is by swiveling the back handle 820 about the first fastener 822 and pulling the back handle 820 away from the first fastener 822 to free it from the first fastener 822. In this implementation, the second fastener attachment point 834 of back handle 820 does not fully enclose around the second fastener 824. Instead, a portion has been removed to provide a crescent cut out sufficiently large so the back handle 820 can be freed from second fastener 824 when the bottom 828 is pushed away from the second fastener 824. The first fastener attachment point 836 also preferably has a slit or cut out to allow the back handle 820 to be pulled from the first fastener 822. To place the back handle 820 in another position, the reverse actions are performed where the first fastener attachment point 836 is pushed into the desired position on the first fastener 822 and the bottom 828 is swiveled toward and snapped into the corresponding position on the second fastener 824 at the second fastener attachment point 836. Preferably, the different positions or notches on the first and second fasteners have been optimized to the human anatomy. That is, the positions are designed to accommodate all hand sizes from adult males to children.

In one implementation, the patient may place his thumb on the front handle 830 and his other fingers on the back handle 820, as shown in FIG. 9. As the patient increases his grip between the back handle 820 and the front handle 830, the force applied is transferred via the first and second fasteners 822 and 824 to deflect the ends of the load sensor 808, which prompt the load sensor 808 to produce an analog signal (e.g., voltage) proportional with the increasing grip strength. The back handle 820 may include fitted depressions such that the patient's fingers may fit within designated spaces on the strength and dexterity testing module 800.

As shown by FIG. 7, the testing module 800 is coupled to the pegboard 702 via the device holder 714. The peg holes of the pegboard 702 may have the sensor mechanism as described with respect to testing module 100 to detect whether an object is in a hole. Device holder 714 has locking mechanism 718 to hold testing module 800 in place. Display panel 710 may be similarly connected to pegboard 702 via device holder 716. Device holder 714 also allows for the vertical adjustment of the testing module 800. The pegboard 702 may also allow for the horizontal adjustment of the device holder and the device being held as discussed with respect to system 200.

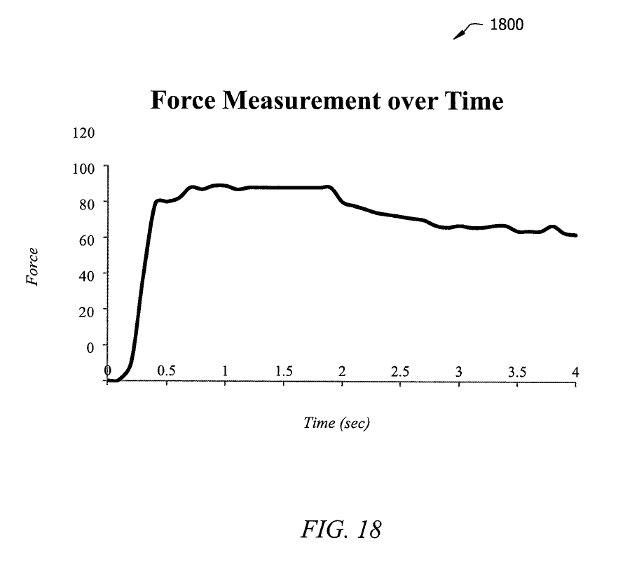

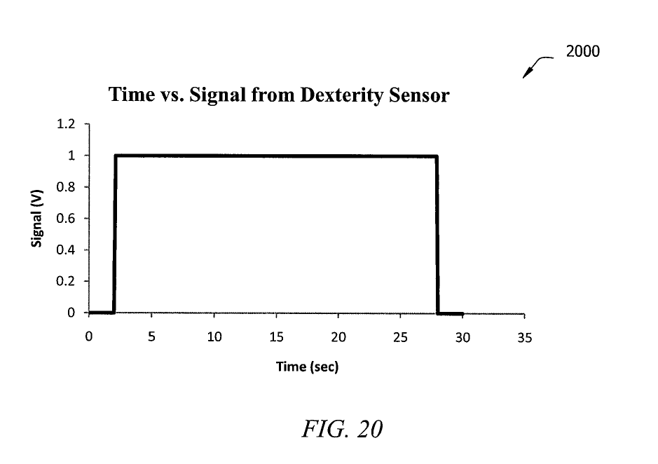

Referring to FIGS. 7 and 8, the display 810 of this implementation of the testing module 800 may be smaller than the display panel 710. The display 810 may display any force number determined by the microprocessor and/or warning messages. The microprocessor of testing module 800 may transmit data to be displayed and/or stored on display panel 710. Accordingly, the display panel may display larger images or graphs that are described in the present disclosure. For example, the display 710 may provide a real-time bio-feedback to the patient as shown in FIG. 23 or the graphs described in FIGS. 18 and 20. The transmission of data can be wireless via the various wireless communication methods described above or wired via communication lines embedded in pegboard 702. In one implementation, a testing module is designated to communicate with its respective display panel 710. In another implementation, more than one testing module 100 or 800 may communicate with and transmit data to a display panel, where the display panel is configured to recognize the transmitting testing module and store or display the data accordingly. In yet another implementation, the testing module provides available display panels to which data can be transmitted, and the user can select the desired display panel. In addition, the pegboard 702 may be connected to an electrical outlet or other similar power source so it can serve as a charging station and/or to provide energy to operate the testing device 800 and/or the display panel 710. The energy may be provided via A/C, USB, or other similar connections. As shown, display panel 710 has a USB port 720 to allow for additional storage space of data and/or transfer of the data that is stored on display panel 710. Another advantage of this implementation is that it provides a versatile mobile testing module separate from the base unit that

As further discussed in the following paragraphs, the present disclosure provides for methods to use the strength and dexterity testing modules and/or strength and dexterity systems in accordance with the present disclosure to administer various tests to measure a patient's muscle hand strength and dexterity. FIG. 9 illustrates how a patient may apply and generate grip strength to be measured by the testing module 800. Similarly, FIG. 10 it illustrates a perspective view of a patient's hand performing a grip strength test with the strength and dexterity testing module 100 in accordance with one or more implementations of various techniques described herein. The grip tests may include a 5-position hand grip test and a rapid exchange grip test. Referring to FIGS. 1 and 10, the 5-position test may include having the patient begin the grip test with his right hand, and the back handle 120 is positioned in the holes of the fasteners 122 and 124 that may be closest to the back plate 132. The patient may be requested to place the fingers on the front handle 130 and thumb next to the back handle 120 and squeeze both handles 120 and 130. Next, the patient's left hand is tested in the same position. The back handle 120 is then repositioned and fitted in another hole in the fasteners 122 and 124 that may be further away from the back plate 132 as compared to its previous position. The grip test is then performed by the patient with his right hand and then his left hand. This procedure may continue until the grip test has been performed for all of the positions on the fasteners 122 and 124.

Referring to FIG. 9, this test can be similarly performed on testing device 800 where the patient begin the grip test with his left hand, and the back handle 820 is positioned in the holes of the fasteners 122 and 124 that may be closest to the front handle 830. The patient may be requested to place the fingers on the back handle 820 and thumb next to the front handle 830 and squeeze both handles 820 and 830. Next, the patient's right hand is tested in the same position. The back handle 820 is then repositioned and fitted in another notch in the fasteners 822 and 824 that may be further away from the front handle 830 as compared to its previous position. The grip test is then performed by the patient with his left hand and then his right hand. This procedure may continue until the grip test has been performed for all of the positions on the fasteners 822 and 824.

With respect to obtaining a patient's maximum effort, a curve plotted with the position of the back handle 120 or 820 (closest to farthest) on the X axis and the force on the Y axis should be a modified bell-shaped curve. A patient's maximum effort is considered to be the maximum amount of force that a patient can exert with the muscle of interest. With feigned weakness in the muscle of interest, the curve will not look like a modified bell-shaped curve. Feigned weakness occurs when the patient gives less than a maximum amount of by deliberately reducing the force he generates with the muscle of interest. In one implementation, the curves described above may be plotted by the microprocessor/circuit board 112 using the force data pertaining to the muscle of interest received from the first load sensor 108 or 116 or load sensor 808.

Also referring to FIGS. 1, 9, and 10, in rapid exchange testing, the position of the back handle 120 or 820 that generated the highest force in the 5-position test should be used. The patient may use the strength and dexterity testing module 100 or 800 for grip testing in one hand. The patient may then generate his maximum effort for the grip test in that hand and then the patient may switch to his other hand. After the patient performs 5-8 grips in each hand, the test is usually complete. The microprocessor/circuit board 112 may automatically determine the force generated from the second load sensor 116 or load sensor 808 for each grip test. The microprocessor/circuit board 112 may then store the force information for each grip test in a memory or display the force information on the display. While FIGS. 9 and 10 have been described with respect to testing modules 100 and 800, respectively, it should be noted that other implementations of the strength and dexterity testing module, which have been described above, can be similarly used by a patient. The above description of the use of the strength and dexterity testing modules 100 and 800 is provided to serve as an example of the use of an exemplary implementation of the strength and dexterity testing module and is not intended to limit the scope of the claims.

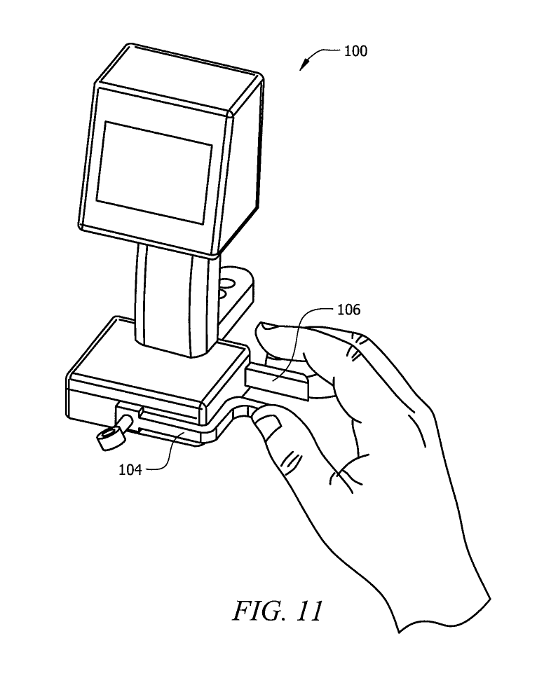

FIG. 11 illustrates a perspective view of a patient's hand performing a pinch test with the strength and dexterity testing module 100 in accordance with one or more implementations of various techniques described herein. The patient perspective view demonstrates how a patient may test his pinch strength. Referring to FIGS. 1 and 11, a patient can apply force to the pinch strength test by placing the thumb at the first component 104 and the index finger at the second component 106 and squeeze the first and second components 104 and 106 together. The squeezing action should prompt the first load sensor 108 to generate an electric signal corresponding to the force applied to the microprocessor board 112. The test can be repeated with the other fingers and the other hand, e.g., left hand, where the thumb would be placed at the second component 106, and the index finger would be at the first component 104. The pinch strength tests may include, but are not limited to, key pinch, tip pinch and palmar pinch. The microprocessor/circuit board 112 may automatically determine the force generated from the first load sensor 108 for each pinch test. The microprocessor/circuit board 112 may then store the force information for each pinch test in a memory or display the force information on the display.

The testing module 800 can be similarly used to perform the pinch tests with the bottom 828 of the back handle 820 and the bottom 832 of the front handle 830 serving as the corresponding first and second components of testing module 100. As shown by FIGS. 9-11, the strength and dexterity testing unit 100 or 800 may be used separate from the pegboard (e.g., 202) of a strength and dexterity system (e.g., 200). While FIG. 8 has been described with respect to testing module 100, it should be noted that other implementations of the strength and dexterity testing module, e.g., testing module 800, which have been described above, can be similarly used by a patient. The above description of the use of the strength and dexterity testing module 100 is provided to serve as an example of the use of an exemplary implementation of the strength and dexterity testing module and is not intended to limit the scope of the claims.

As discussed previously, the testing module 100 alone, or the strength and dexterity system 200 integrating the testing module 100, can be used to measure intrinsic muscle hand strength. There are several different types of intrinsic muscles, and the following description serve as examples of the way certain intrinsic muscles can be measured and is not intended to limit the scope of the claims. It is understood that various implementations of the testing unit and strength and dexterity system can be used in similar manners.

Referring to FIGS. 1, 3, and 12, for Hypothenar Muscle (small finger abduction) testing, which is illustrated on FIG. 12, the patient's hand is placed palm down (prone) on the pegboard 302 with all of his fingers except for his thumb inside the loop such that his smallest finger is farthest away from the first load sensor 108. The loop is placed at the smallest finger's proximal interphalangeal joint. Restraining pegs 322 are placed on the ulnar side of the ring finger distal interphalangeal joint, 102 cm proximal to the radial and ulnar styloid at the wrist and along the radial and ulnar border of the forearm at the most proximal peg holes available. The pegs 322 may be used to restrain the forearm such that the forearm may be positioned close enough to restrain the motion of the forearm during testing, but not to cause discomfort. In one implementation, pegs 322 should be placed such that the patient's bony prominences are avoided. After positioning the patient's hand on the pegboard 302, the patient may be instructed to pull his small finger against the loop or away from the first load sensor 108. The first load sensor 108 may measure the force applied by the patient and may send a signal representing its measurement to the microprocessor/circuit board 112 as described above.

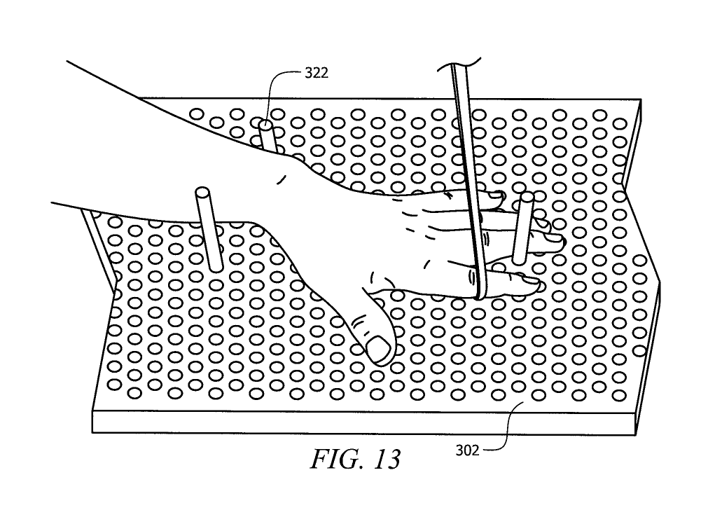

Referring to FIGS. 1, 2, and 13, for First Dorsal Interosseous Muscle (index finger abduction) testing, which is illustrated on FIG. 13, the patient's hand is placed palm down (prone) on the pegboard 202 with all of his fingers except for his thumb inside the loop such that the index finger is farthest away from the first load sensor 108. The loop is placed at the index finger proximal interphalangeal joint. Restraining pegs are placed on the radial side of the middle finger distal interphalangeal joint, 102 cm proximal to the radial and ulnar styloid at the wrist and along the radial and ulnar border of the forearm at the most proximal peg holes available. The restraining pegs for the patient's forearm should be snug enough to not allow motion of the forearm during testing, but not so tight as to cause discomfort. Again, pegs should be placed such that bony prominences are avoided for comfort. The patient may then be instructed to pull with his index finger against the loop or away from the first load sensor 108. The first load sensor 108 may measure the force applied by the patient and may send a signal representing its measurement to the microprocessor/circuit board 112 as described above.

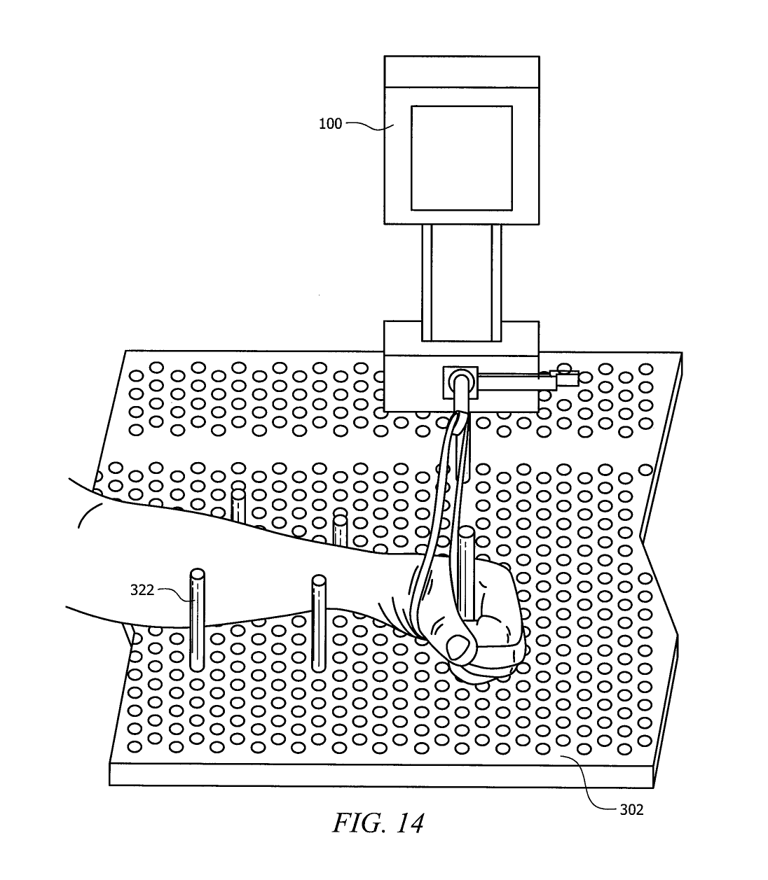

For Abductor Pollicis Brevis Muscle (thumb palmar abduction) testing which is illustrated on FIG. 14, the patient's hand is rested on its ulnar border with the forearm in neutral prono-supination. Referring to FIGS. 1, 2, and 14, the back of the hand should face the first load sensor 108. The strength and dexterity testing module 100 may be moved up the vertical axis so that the eyebolt 102 is level with the thumb and the loop is placed around the thumb metacarpophalangeal joint. A restraining peg is placed in line with the distal palmar crease, and the patient may flex his fingers around this peg. The wrist is positioned in 10 degrees of extension and restraining pegs are placed both volar and dorsal to the wrist at the level of the radial styloid. Two more pegs, one dorsal and one volar are placed proximally along the forearm at the most proximal peg holes available. The forearm pegs should be snug enough to not allow motion of the forearm during testing, but not so tight as to cause discomfort. The patient is then instructed to palmarly abduct his thumb. The first load sensor 108 may measure the force applied by the patient and may send a signal representing its measurement to the microprocessor/circuit board 112 as described above.

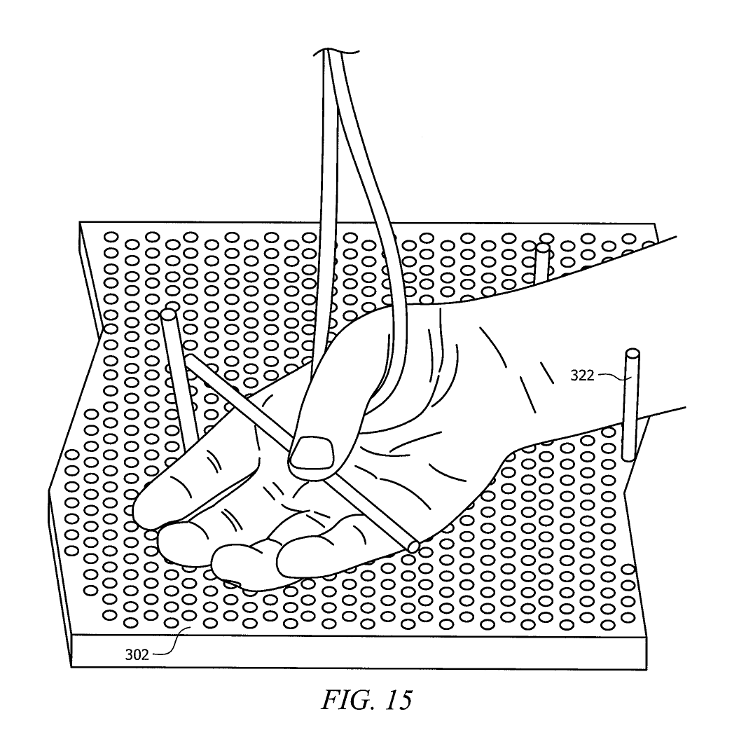

For Opponens PollicisMuscle (thumb opposition) testing which is illustrated in FIG. 15, the hand is resting on the pegboard 302 palm up with the forearm in supination. Referring to FIGS. 1, 2, and 15, the radial border of the hand faces the first load sensor 108. The strength and dexterity testing module 100 may be adjusted on the vertical axis so that the eyebolt 102 (not shown in FIG. 15) is level with the thumb and the loop is placed at or slightly proximal to the thumb metacarpophalangeal joint. Pegs 322 are placed along the forearm to minimize excess movement as previously described. A horizontal peg is also placed in the patient's palm to restrain any vertical movement by the patient's hand. The patient is instructed to touch the tip of his thumb to the tip of his small finger. A clinician may restrain the patient's four fingers, allowing only the patient's thumb to move. The first load sensor 108 may measure the force applied by the patient and may send a signal representing its measurement to the microprocessor/circuit board 112 as described above.

While FIGS. 12-15 have been described with respect to testing module 100, it should be noted that other implementations of the strength and dexterity testing module, e.g., testing module 800, which have been described above, can be similarly used by a patient to obtain IHMS measurement. The above description of the use of the strength and dexterity testing module 100 is provided to serve as an example of the use of an exemplary implementation of the strength and dexterity testing module and is not intended to limit the scope of the claims.

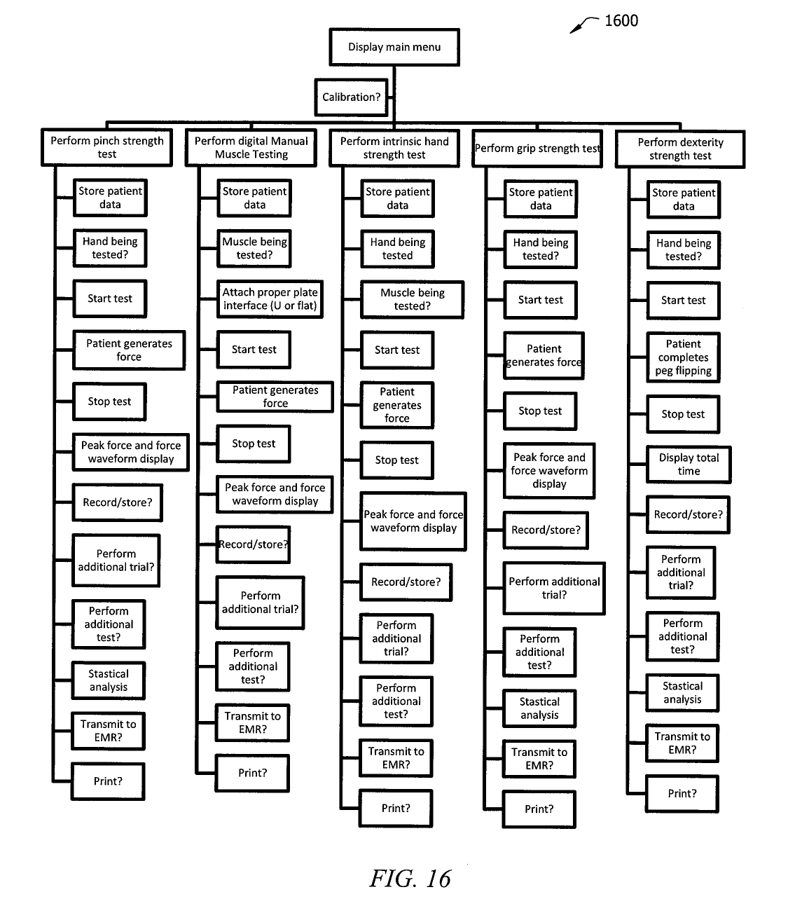

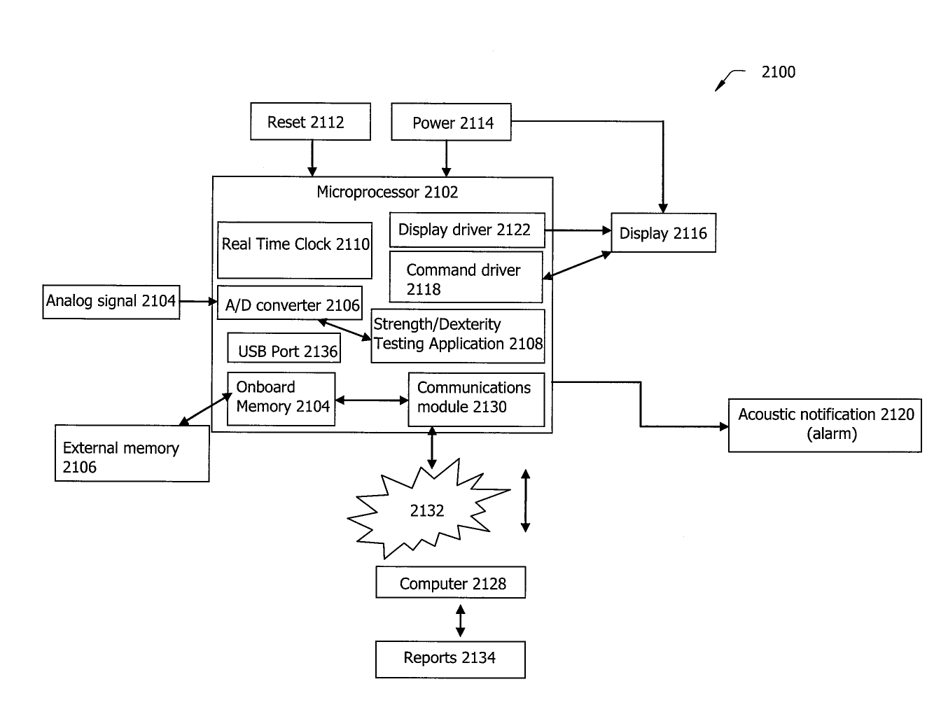

FIG. 16 illustrates a flow diagram of a method 1600 for testing strength and dexterity in accordance with one or more implementations of various techniques described herein. In one implementation, the method 1600 for testing strength and dexterity may include displaying a main menu for a strength and dexterity testing application 2108. The strength and dexterity testing application 2108 will be described later with reference to FIG. 21. In one implementation, the main menu may allow for a calibration option. Referring to FIGS. 1, 2, and 16, the calibration option may be used to calibrate the load sensors 108 and 116 or the different ends of the load sensor 808.

The main menu may list each test that may be performed on the strength and dexterity system, e.g., system 200 of FIG. 2 or 700 of FIG. 7. In one implementation, a clinician may select a test (i.e., intrinsic, pinch, grip manual muscle or dexterity) to perform from a list of tests provided on the main menu. As previously discussed, this option is not necessary in operating the testing module 800 described in FIGS. 7 and 8 because the load sensor 808 is configured to “know” which test is being performed.

Referring to FIGS. 1, 2, and 16, after selecting the test to perform, the clinician may then input the patient data into the system such that it may be stored on the system 200 or 700. The patient data may include the hand and muscle that will be tested. In one implementation, the patient data may also include the vitals of the patient such as weight, height and similar information. The clinician may then initiate the selected test, and the patient may generate a force required for the corresponding test. The force may be detected by the load sensors 108, 116, or 808 and sent to the respective microprocessor/circuit board, e.g., 112. Referring to FIGS. 16 and 21, after the test is stopped by the clinician, the strength and dexterity testing application 2108 may determine a peak force, a waveform graph and other parameters. The strength and dexterity testing application 2108 may display the peak force, the waveform graph and other parameters on the display. The clinician may then store the data or reject the test. The test may be repeated as the clinician may deem necessary. After storing the data, the strength and dexterity testing application 2108 may perform a statistical analysis on the data. In another implementation, the strength and dexterity testing application 2108 may send the data to a remote computer to perform the statistical analysis on the data. The statistical analysis may include computing a mean, a coefficient of variance, a standard deviation or percentile calculations compared to the norms.

Further, the strength and dexterity testing application 2108 may perform a validity analysis for the rapid exchange testing (grip). The validity analysis may compare the patient's actual test data pattern with an expected test data pattern to determine whether the patient's actual test data was valid. In one implementation, the validity analysis may analyze a coefficient of variation for the patient's actual test data or between the patient's actual test data and the expected test data to determine whether the patient's actual test data was a valid, equivocal or invalid. In one implementation, the coefficient of variation that is less than or equal to 15% may be valid, a coefficient of variation that is between 15.1%-20% may be equivocal, and a coefficient of variation greater than 20% may be invalid.

The validity analysis may be performed by analyzing the patient's data related to simple repetitions of an intrinsic hand muscle strength test, a pinch strength test or a grip strength test. For grip strength validity analysis, the coefficient of variance from 0-10% is considered valid, 10.1-15% is considered equivocal and greater than 15% is considered invalid. For pinch validity, the coefficient of variance from 0-15% is considered valid, 15.1-20% is considered equivocal and greater than 20% is considered invalid. The validity analysis may include calculating a coefficient of variation for several exertions. The validity data may then be transmitted via a communications module on the microprocessor/circuit board 112 to a remote or secure computer or display panel 710 in FIG. 7. As previously discussed, the communications module may employ Bluetooth or Wi-Fi technology to transmit data or wired means known to those skilled in the art. In another implementation, the clinician may choose to print the validity data or any other data recorded by the strength and dexterity testing application. In one implementation, the microprocessor/circuit board 112 and/or display panel 710 of FIG. 7 may include a USB port such that a printer may be connected to the microprocessor/circuit board 112 and a print option may be available.

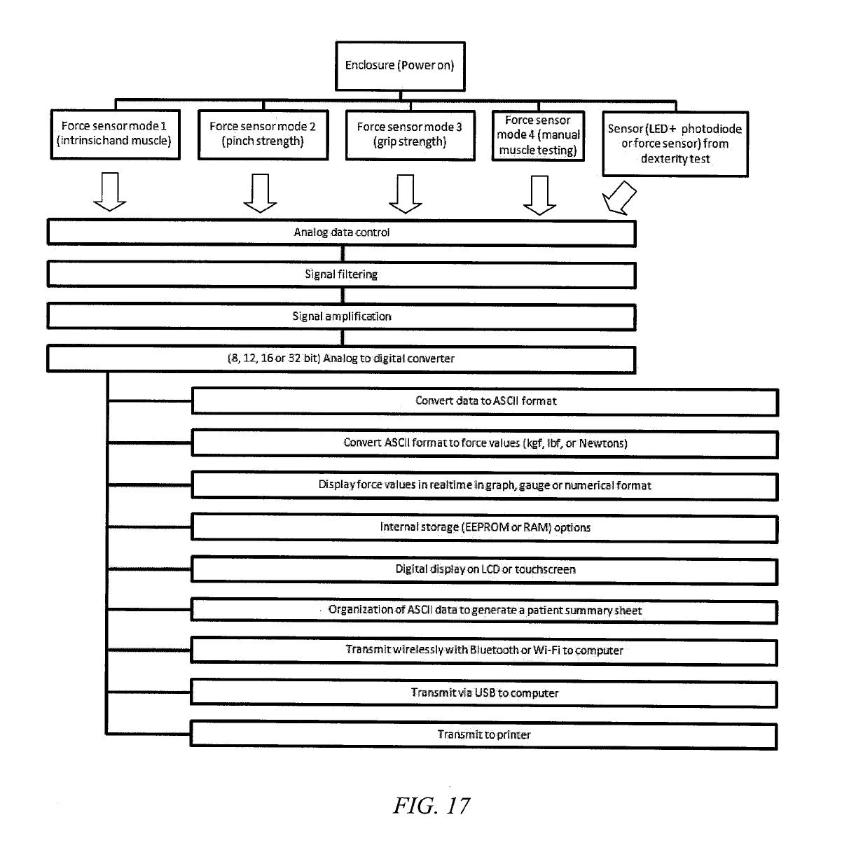

FIG. 17 illustrates a flow diagram of a method for processing signals obtained from the strength and dexterity testing in accordance with one or more implementations of various techniques described herein. In one implementation, the method for processing signals obtained from the strength and dexterity testing includes a signal generation. When the strength and dexterity system is powered on, each of the load sensors or sensors instrumented in the peg holes may generate a voltage signal. Referring to FIGS. 1, 2, and 17, in one implementation, the voltage signal may be input into the microprocessor/circuit board 112 such that the analog voltage signal may be filtered for disruptions such as 60 Hz noise. The voltage signal may then be amplified via instrumentation amplifiers on the microprocessor/circuit board 112. In one implementation, the microprocessor/circuit board 112 may include an analog to digital converter such that the signals from the sensors may be converted to ASCII digital data. The strength and dexterity application 1808 may convert the raw numbers to force values. These force values may be displayed on the display by the microprocessor/circuit board 112. Internal storage via RAM or EEPROM on the microprocessor/circuit board 112 may be used to store recorded force values. Furthermore, the values generated by testing can be used to create a patient summary sheet for integration with health information systems or printed out to be placed in a patient chart. This may occur directly on the microprocessor/circuit board 112 or sent via the communication module to a third party computer at a remote location for further analysis.

While FIGS. 16-17 have been described with respect to testing module 100, it should be noted that the method also applies to other implementations of the strength and dexterity testing module, e.g., testing module 800, which have been described above. The above description of the use of the strength and dexterity testing module 100 is provided to serve as an example of the use of an exemplary implementation of the strength and dexterity testing module and is not intended to limit the scope of the claims.

FIG. 18 illustrates an example of a force diagram generated by a load sensor from the strength and dexterity testing in accordance with one or more implementations of various techniques described herein. In one implementation, the force diagram 1800 may be generated by a patient exerting grip strength, intrinsic, grip or manual muscle strength. The slope and area under the curve of this force diagram may be used to derive additional parameters like hand grip validity, rapid exchange grip (REG) testing or time until fatigue. Referring to FIGS. 1, 2, 8, and 18, in one implementation, the force diagram may be output by the microprocessor/circuit board 112 based on the signals received from the load sensor 108, 116, or 808. In one implementation the diagram 1800 may be displayed on display panel 710 of FIG. 7.

FIG. 19 illustrates a flow diagram of an automated method for measuring dexterity in accordance with one or more implementations of various techniques described herein. In one implementation, when a patient lifts a first peg during a test, the change in lighting inside the peg hole may activate a light sensor located inside the peg hole. The light sensor may produce a signal that may be sent to the microprocessor/circuit board 112 and undergo similar signal conditioning as the load sensors 108 and 116. For instance, the microprocessor/circuit board 112 may amplify and filter the light signal. When the microprocessor/circuit board 112 receives a light signal indicating that the patient has lifted the first peg, the strength and dexterity testing application 1808 may start a timer. In one implementation, the patient may be required to move a predetermined number of pegs from one peg hole to another. After the patient finishes the test and moves the final peg to the final peg hole, and after the microprocessor/circuit board 112 receives the final light signal from the light sensor, the microprocessor/circuit board 112 may stop the timer. In one implementation, the microprocessor/circuit board 112 may automatically calculate the time difference between when the first peg was removed from the first peg hole and when the final peg was placed in the final peg hole. In another implementation, the clinician may manually start and stop the timer. This mechanism may be incorporated within the pegboard 202 or as an additional smart pegboard attachment that couples to the bottom of the pegboard 202. The microprocessor/circuit board 112 may include a real time clock such that it may record a time stamp indicating when a peg is removed from the peg hole and/or when a peg is inserted in the peg hole.

FIG. 20 illustrates a time versus signal graph generated by the strength and dexterity system in accordance with one or more implementations of various techniques described herein. In one implementation, the graph 2000 indicates that when the dexterity test began (i.e., when the first peg was removed) and when the dexterity test was completed (i.e., when the final peg was placed in the peg hole). Referring to FIGS. 1, 2, and 20, in one implementation, the light signal may be received by the microprocessor/circuit board 112 as a voltage. As such, when the microprocessor/circuit board 112 senses or receives the voltage, the microprocessor/circuit board 112 may initiate the timer. When the light signal is removed or when the voltage from the light signal falls back to 0, the microprocessor/circuit board 112 may stop the timer and calculate the time difference. According to the graph 2000, the light signal was initially received by the microprocessor/circuit board 112 at 2.5 second and the light signal was removed at 27.5 seconds. In this graph, the light signal provides a 1 volt signal to the microprocessor/circuit board 112. In one implementation the diagram 20000 may be displayed on display panel 710 of FIG. 7. In another implementation, the diagram 2000 may be displayed on display 110 of FIG. 1.

FIG. 21 illustrates a schematic diagram of a microprocessor system 2100 in accordance with one or more implementations of various techniques described herein. For instance, this microprocessor system 2100 may be used in testing module 100 and/or 800. In one implementation, the microprocessor 2102 may correspond to the microprocessor/circuit board 112 as described in FIG. 1. The analog signal 2104 may represent a signal from the load sensors 108, 116 or the light signal sensors described above with respect to the peg holes. The analog signal may be converted to a digital signal by an analog-to-digital (A/D) converter 2106. The digital signal may then be processed by the strength and dexterity application 2108 for analysis. The microprocessor 2102 may also include a real time clock 2110 which may allow the microprocessor 2102 to accurately track the times at which the analog signal 2104 may have been received by the microprocessor. In one implementation, the real time clock 2110 may be used as the timer described above.

Referring to FIG. 21, a reset function 2112 may restart a preloaded program (i.e., the strength and dexterity application 2108) and delete any stored data on the memory of the microprocessor 2102. A power source 2114 may be coupled to the microprocessor 2102 and the display 2116, which can correspond to display 110 shown in FIG. 1 or a separate display coupled to either a testing unit, e.g., 100 of FIG. 1, or a pegboard, e.g., 202 of FIG. 2. The display 2116 may present graphics that are related to the strength and dexterity testing application 2108. The display 2116 may be an LCD or any other display type that is capable of presenting digital graphics. In one implementation, the display 2116 may be a touch screen such that it may receive inputs from a patient. The touch screen display 2116 may send commands back to the microprocessor 2102 through a command driver 2118.

An acoustic notification 2120 may be coupled to the microprocessor 2102 in the form of an alarm or beep. The acoustic notification 2120 may be integrated with the microprocessor 2102 to provide acoustic feedback based on the inputs provided to the microprocessor 2102 via the display 2116 (e.g., touchscreen) or the like. Furthermore, the acoustic notification 2120 may warn against excessive force which may damage the load sensors. In addition, the acoustic notification 2120 may be used to notify a patient that he has reached a certain plateau of strength or recovery while the strength and dexterity testing is being performed. The display driver 2122 may allow the microprocessor 2102 to send force signals to the display 2116 in an appropriate format such that the display 2116 may indicate the force being applied by the patient.

Referring to FIG. 21, The microprocessor 2102 may store signal, validity, or any other data to either onboard memory 2124 or external memory 2126 (FLASH, EEPROM, CD-ROM, DVD-ROM, etc) as necessary. As shown, USB Port 2136 provides one way to transmit this data to a storage component. The data from the microprocessor 2102, onboard memory 2124 or external memory 2126 may also be sent to an external computer 2128 via a communications module 2130. As previously discussed, the communications module 2130 may be wired or wireless, e.g., a Bluetooth or RF (radio frequency) radio or any other similar communications means such that data may be sent from the microprocessor. In one implementation, the communications equipment may broadcast the signal from the microprocessor 2102, the onboard memory 2124 or the external memory 2126 through a network 2132 to a computer 2128. The computer 2128 may include a third-party computer, laptop or any other similar device that may be capable of receiving a signal from the communication module 2130. The computer 2128 may then generate reports 2134 based on the data stored on the onboard memory 2124 or the external memory 2126. The reports 2134 may include patient summary reports, data logs and other useful information pertaining to the strength and dexterity testing. In one implementation, the reports 2134 may be transferred to a printer or any other similar media.

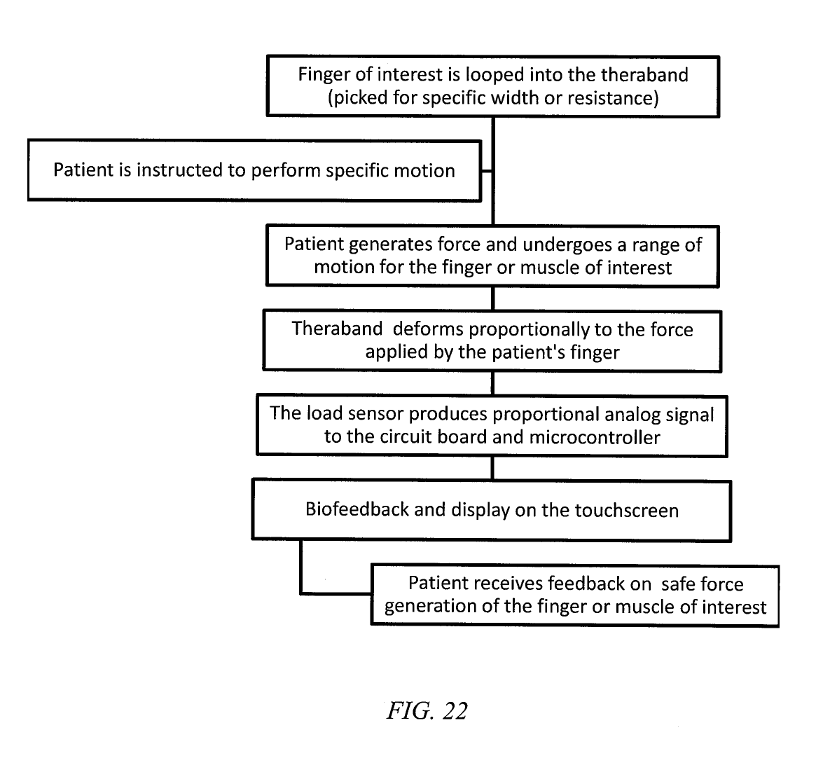

FIG. 22 illustrates a flow diagram of a method for performing a strength and dexterity test using a Theraband® in accordance with one or more implementations of various techniques described herein. In one implementation, the clinician may loop the patient's finger of interest into the Theraband®. The Theraband® may replace the additional attachment 602 as shown in FIG. 6. In another implementation, the Theraband® may be connected to force transfer member 704 shown in FIG. 7. The Theraband® may be a specific width in order to create a specified resistance. After looping the finger of interest into the Theraband®, the patient may perform a specific motion as defined by the clinician. When the patient performed the specific motion, the patient may generate a force and undergo a range of motion for the finger or muscle of interest. As a result, the Theraband® may deform in proportion to the force applied by the patient's finger. The load sensor produces proportional analog signal to the microprocessor. The display, either of the testing module or the separate display panel, may display the forces calculated or selected to be displayed by the user. In another implementation, biofeedback such as that shown in FIG. 23 may be displayed. The patient then has a feedback of the force applied.

Referring to FIGS. 1, 6, 8, and 23, the first load sensor 108 or load sensor 808 may detect the force applied by the patient's finger and produce an analog signal that may represent the applied force. The analog signal may be sent to the microprocessor/circuit board 112 as described above. The microprocessor/circuit board 112 may analyze the analog signal and display the applied force on the display 110 and/or 710 of FIG. 7. In one implementation, the display 110 and/or 710 may provide a real-time bio-feedback to the patient as shown in FIG. 23. In FIG. 23, there are three zones within which the force being applied can fall. The first zone is indicated as “GOOD,” the second zone as “WARNING,” and the third zone as “DANGER.”The real-time bio-feedback may display a gauge indicating the force applied by the patient. The gauge may indicate force levels that are safe and dangerous so that patient's may maximize his efforts within an effective force range that prevents over-exertion and reduces the likelihood of damaging a muscle/tendon unit being rehabilitated. Additionally, the display can aid the patient by displaying proper technique for a given exercise, number of repetitions performed, elapsed time and maximum force.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A device comprising:
a strength testing unit comprising:
a force sensor;
a front handle configured to receive the force sensor;
a first fastener;
a second fastener, wherein the first and second fasteners are coupled to the force sensor;
a back handle coupled to the first and second fasteners and positioned at a distance away from the front handle;
a force transfer member coupled to the back handle,
wherein the force sensor is configured to generate one or more electric signals corresponding to the force applied to at least one of the front and back handles; and
a processing unit coupled to the force sensor, the processing unit configured to determine a measurement of the applied force and to display the measurement on a first display coupled to the processing unit.

2. The device of claim 1 further comprising:
a board comprising:
one or more holes;
one or more sensors coupled to the processing unit, the one or more sensors configured to detect whether one or more objects are inside the one or more holes and to generate one or more electric signals in response to said detection; and
one or more openings configured to receive the strength testing unit.

3. The device of claim 2 further comprising:
a second display configured to communicate with the processing unit to at least receive and display information transmitted from the processing unit; and
a data storage unit configured to communicate with the processing unit to at least receive and store information transmitted from the processing unit.

4. The device of claim 2 further comprising a timing unit coupled to the processing unit, the timing unit configured to measure the time elapsed between at least two electric signals generated by the one or more sensors.

5. The device of claim 1 wherein the processing unit determines a type of test being administered based, at least in part, on the type of force being applied to the force sensor.

6. The device of claim 1 wherein the processing unit provides to a user information associated with a force being applied to the device by the user, said information comprising an effective range of force to allow the user to maximize the force being applied to the device within said range.

7. A device comprising:
a strength testing unit comprising:
a pinch tester comprising:
a first component coupled to a first force sensor;
a second component, wherein the second component is immobile;
an intrinsic muscle tester comprising:
a force transfer member configured to fit on or around one or more digits of a user's hand, wherein the force transfer member is coupled to said first force sensor,
wherein the first force sensor is configured to generate one or more electric signals corresponding to the force applied to at least the first component of the pinch tester or the intrinsic muscle tester; and
a processing unit coupled to the first force sensor, the processing unit configured to determine a measurement of the applied force and to display the measurement on a display coupled to the processing unit.

8. The device of claim 7 further comprising:
a grip tester comprising:
a front component coupled to a second force sensor, wherein the second force sensor is configured to generate one or more electric signals corresponding to the force applied to at least the front component; and
a back component located a distance away from the front component, said distance is adjustable.
wherein the processing unit is configured to determine a measurement of the force applied to at least the front component and to display the measurement on the display.

9. The device of claim 8 further comprising:
a board comprising:
one or more holes;
one or more sensors coupled to the processing unit, the one or more sensors configured to detect whether one or more objects are inside the one or more holes and to generate one or more electric signals in response to said detection; and
one or more openings configured to receive the strength testing unit.

10. The device of claim 9 further comprising a timing unit coupled to the processing unit, the timing unit configured to measure the time elapsed between at least two electric signals generated by the one or more sensors.

11. The device of claim 7 wherein the processing unit is configured to convert the received electric signals to one or more corresponding force measurements.

12. The device of claim 7 further comprising a data storage unit coupled to the processing unit, the data storage unit configured to store at least the one or more corresponding force measurements.

13. The device of claim 9 wherein the one or more sensors are configured to detect photons.

14. The device of claim 7 wherein the processing unit is configured to transmit the one or more force measurements to a receiving unit at remote location.

15. A method comprising the steps of
providing a device comprising:
a strength testing unit comprising:
a force sensor
a front handle configured to receive a force sensor;
a first fastener;
a second fastener, wherein the first and second fasteners are coupled to the force sensor;
a back handle coupled to the first and second fasteners and positioned at a distance away from the front handle;
a force transfer member coupled to the back handle,
allowing a user to apply force to at least the back handle, said force being transferred from the back handle to the force sensor via the first and second fastener;
generating one or more electric signals corresponding to the force applied, said one or more signals generated by the force sensor;
determining a measurement of the force applied, said measurement determined by a processing unit coupled to the first force sensor; and
displaying the measurement on a display coupled to the processing unit.

16. The method of claim 15 further comprising the steps of:
detecting whether one or more objects are inside one or more holes on a board, said detecting is achieved by one or more sensors coupled to the one or more holes;
generating one or more electric signals in response to said detection, said generating is achieved by the one or more sensors; and
receiving the one or more signals by the processing unit, said processing unit is coupled to the one or more sensors.

17. The method of claim 16 further comprising the step of:
providing a time stamp to indicate when the one or more objects are inserted or removed from the one or more holes.

18. The method of claim 15 further comprising the step of:
determining by the processing unit a type of test being administered based, at least in part, on the type of force being applied to the force sensor.

19. The method of claim 15 further comprising the steps of:
restraining the user's hand to isolate the portion of the hand to be tested; and
connecting the isolated portion of the hand to the force transfer member;
wherein the force applied by the user is a pulling force on the back handle.

20. The method of claim 15 wherein the user applies the force by pushing the back handle inward toward the front handle.

21. The method of claim 15 further comprising the step of
providing to the user information associated with the applied force, said information comprising an effective range of force to allow the user to maximize the force being applied to the device within said range.
